(19) United States
(12) Patent Application Publication
(10) Pub. No.: US 2020/0207823 A1
(43) Pub. Date: Jul. 2, 2020

(54) PROCESS FOR PRODUCING, ISOLATING, AND PURIFYING MODIFIED RECOMBINANT PROTEINS

(71) Applicant: Coherus Biosciences, Inc., Redwood City, CA (US)

(72) Inventors: Stuart Gallant, Redwood City, CA (US); John Ogez, Redwood City, CA (US); Charles Olson, Redwood City, CA (US)

(73) Assignee: Coherus Biosciences, Inc., Redwood City, CA (US)

(21) Appl. No.: 16/728,523

(22) Filed: Dec. 27, 2019

Related U.S. Application Data
(60) Provisional application No. 62/786,142, filed on Dec. 28, 2018.

(51) Int. Cl. C07K 14/535; C12P 21/02; C07K 1/06; C07K 1/16; C07K 1/18; B01D 15/36; B01D 15/38; B01D 61/14; B01D 15/18

(52) U.S. Cl. CPC C07K 14/535; C12P 21/02; C07K 1/06; C07K 1/165; C07K 1/18; B01D 15/363; B01D 2315/16; B01D 15/3847; B01D 61/145; B01D 15/1871; B01D 2311/2626; B01D 2311/2623; B01D 15/362

(57) ABSTRACT

The invention provides for methods and processes for producing, isolating, and purifying modified proteins. In particular, the invention provides for the production, isolation and purification of PEGylated recombinant methionyl human granulocyte colony stimulating factor used for therapeutic purposes.

### CROSS-REFERENCE TO RELATED APPLICATION

This application claims priority to U.S. Provisional Patent Application Ser. No. 62/786,142, filed Dec. 28, 2018, the entire contents of which are herein incorporated by reference.

### RELATED APPLICATIONS AND INCORPORATION BY REFERENCE

All documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention generally relates to the production, isolation, and purification of a modified recombinant protein, specifically a protein used as a diagnostic, therapeutic, or prophylactic agent.

### BACKGROUND OF THE INVENTION

Proteins for therapeutic use are available in suitable forms and in adequate quantities largely as a result of the advances in recombinant DNA technologies. Examples of recombinant therapeutic proteins include but are not limited to erythropoietin (EPO), granulocyte colony-stimulating factor (G-CSF), alpha-galactosidase A, alpha-L-iduronidase (rhIDU; laronidase), N-acetylgalactosamine-4-sulfatase (rhASB; galsulfase), dornase alfa (a Dnase), tissue plasminogen activator (TPA), glucocerebrosidase, interferons (IF s), insulin-like growth factor 1 (IGF-1), and rasburicase (a urate oxidase analog).

The availability of recombinant proteins has fostered advances in protein formulation and chemical modification. Chemical modification can facilitate protein protection as attachment of a chemical moiety may effectively block a proteolytic enzyme from physical contact with the protein backbone itself, and thus prevent degradation. Additional advantages include, but are not limited, to increasing the stability and circulation time of the therapeutic protein and decreasing immunogenicity. Polyethylene glycol (“PEG”) is a chemical moiety which has been used in the preparation of therapeutic proteins. One specific therapeutic protein which has been chemically modified with PEG is G-CSF. G-CSF induces the rapid proliferation and release of white blood cells (e.g., neutrophilic granulocytes) into the blood stream, and thereby provides a therapeutic effect in fighting infection.

Development of successful production, isolation, and purification strategies for such chemically-modified recombinant proteins is challenging because of the problems associated with the scale-up of bioprocesses. The production, isolation, and purification of therapeutic proteins from various source materials involves a number of steps and procedures. These therapeutic proteins may be obtained from plasma or tissue extracts, for example, or may be produced by cell cultures using eukaryotic or prokaryotic cells containing at least one recombinant plasmid encoding the desired protein. The engineered proteins are then either secreted into the surrounding media or into the perinuclear space, or made intracellularly, for e.g., present in inclusion bodies, and extracted from the cells. A number of technologies are utilized for purifying desired proteins from their source material.

Purification processes comprise procedures in which the protein of interest is separated from the source materials on the basis of solubility, ionic charge, molecular size, adsorption properties, and specific binding to other molecules. The procedures include but are not limited to gel filtration chromatography, ion-exchange chromatography, affinity chromatography, hydrophobic interaction chromatography, and mixed-mode chromatography. The disclosed methods address the challenging problem of scaling up the production, isolation, and purification of modified recombinant human G-CSF for therapeutic purposes by providing an upstream process (UP) and a downstream process (DSP) as further described herein.

Citation or identification of any document in this application is not an admission that such document is available as prior art to the present invention.

### SUMMARY OF THE INVENTION

Provided herein are processes and methods for producing, isolating, and purifying PEGylated, recombinant methionyl human granulocyte colony-stimulating factor (r-met-Hu-G-CSF) for therapeutic purposes, preferably on a commercial scale.

Provided herein are methods for producing recombinant methionyl human granulocyte colony-stimulating factor (r-met-Hu-G-CSF) that include: (a) contacting cells comprising a nucleic acid encoding r-met-Hu-G-CSF with a culture medium to create a fermentation medium; (b) fermenting the cells under fed-batch conditions causing the cells to produce r-met-Hu-G-CSF; (c) harvesting the cells from the fermentation medium by centrifugation; (d) lysing the cells harvested from the fermentation medium to release inclusion bodies comprising r-met-Hu-G-CSF; and (e) storing the inclusion bodies. Some embodiments further include, after step (e): suspending inclusion bodies comprising r-met-Hu-G-CSF in a solubilization buffer; (g) oxidizing solubilized r-met-Hu-G-CSF to permit the r-met-Hu-G-CSF to fold and form disulfide bonds; (h) subjecting a product of step (g) to Dowex flow-through chromatography; (i) subjecting a product of step (h) to acid precipitation; (j) subjecting a product of step (i) to anion exchange chromatography; (k) subjecting a product of step (j) to cation exchange chromatography; (l) subjecting a product of step (k) to mixed mode chromatography; (m) concentrating a product of step (l); and (n) exchanging r-met-Hu-G-CSF in the product of step (m) into a buffer by ultrafiltration and diafiltration. Some embodiments of any of the methods described herein further include after step (n): (o) contacting a r-met-Hu-G-CSF with a PEGylation reagent under suitable reaction conditions to PEGylate the r-met-Hu-G-CSF; (p) subjecting a product of step (o) to cation exchange chromatography to remove the reaction by-products from PEGylated r-met-Hu-G-CSF; (q) concentrating a product of step (p); (r) exchanging the PEGylated r-met-Hu-G-CSF in a product of step (q) into a buffer by ultrafiltration and diafiltration; (s) adding a surfactant to a product of step (r); (t) adjusting the pH of a product of step (s) to a target value by adding HCl or NaOH; (u) diluting a product of step (t) with additional diafiltration buffer to achieve a target PEGylated r-met-Hu-G-CSF concentration of 10.0 mg/mL; and (v) subjecting the PEGylated r-met-Hu-G-CSF product of step (u) to 0.2-μm filtration. Dome embodiments of any of the methods described herein further include storing the PEGylated r-met-Hu-G-CSF product at 5±3° C.

Also provided herein are methods for purifying r-met-Hu-G-CSF from inclusion bodies that include: (a) suspending inclusion bodies comprising r-met-Hu-G-CSF in a solubilization buffer; (b) oxidizing solubilized r-met-Hu-G-CSF to permit the r-met-Hu-G-CSF to fold and form disulfide bonds; (c) subjecting a product of step (b) to Dowex flow-through chromatography; (d) subjecting a product of step (c) to acid precipitation; (e) subjecting a product of step (d) to anion exchange chromatography; (f) subjecting a product of step (e) to cation exchange chromatography; (g) subjecting a product of step (f) to mixed mode chromatography; (h) concentrating a product of step (g); and (i) exchanging r-met-Hu-G-CSF in the product of step (h) into a buffer by ultrafiltration and diafiltration. Some embodiments of any of the methods described herein further include after step (i): (j) contacting a r-met-Hu-G-CSF with a PEGylation reagent under suitable reaction conditions to PEGylate the r-met-Hu-G-CSF; (k) subjecting a product of step (j) to cation exchange chromatography to remove the reaction by-products from PEGylated r-met-Hu-G-CSF; (1) concentrating a product of step (k); (m) exchanging the PEGylated r-met-Hu-G-CSF in a product of step (1) into a buffer by ultrafiltration and diafiltration; (n) adding a surfactant to a product of step (m); (o) adjusting the pH of a product of step (n) to a target value by adding HCl or NaOH; (p) diluting a product of step (o) with additional diafiltration buffer to achieve a target PEGylated r-met-Hu-G-CSF concentration of 10.0 mg/mL; and (q) subjecting the PEGylated r-met-Hu-G-CSF product of step (p) to 0.2-μm filtration.

Also provided herein are methods of producing a PEGylated and purified r-met-Hu-G-CSF that include: (a) contacting a r-met-Hu-G-CSF with a PEGylation reagent under suitable reaction conditions to PEGylate the r-met-Hu-G-CSF; (b) subjecting a product of step (a) to cation exchange chromatography to remove the reaction by-products from PEGylated r-met-Hu-G-CSF; (c) concentrating a product of step (b); (d) exchanging the PEGylated r-met-Hu-G-CSF in a product of step (c) into a buffer by ultrafiltration and diafiltration; (e) a surfactant to a product of step (d); (f) adjusting the pH of a product of step (e) to a target value by adding HCl or NaOH; (g) diluting a product of step (f) with additional diafiltration buffer to achieve a target PEGylated r-met-Hu-G-CSF concentration of 10.0 mg/mL; and (h) subjecting the PEGylated r-met-Hu-G-CSF product of step (g) to 0.2-μm filtration. Some embodiments of any of the methods described herein further include, after step (h), storing the PEGylated r-met-Hu-G-CSF product at 5±3° C.

Also provided herein is an upstream process (UP) which includes, but is not limited to, the following steps: Product Fermentation, Cell Harvest, Cell Lysis, and Inclusion Body Harvest and Wash. In some embodiments of any of the processes described herein, Product Fermentation is preceded by Primary Inoculum preparation.

Also provided herein is a downstream processes which includes, but is not limited to, the following steps: Inclusion Body Thaw/Solubilization/Oxidation, Dowex Chromatography, Acid Precipitation/Clarification, Anion Exchange Chromatography, Cation Exchange Chromatography, Mixed-mode Chromatography, UF/DF, PEGylation, and Bulk Formulation and Fill. In some embodiments of any of the processes described herein, the downstream process further includes a second Cation Exchange Chromatography step and a final UF/DF.

In particular embodiments of any of the processes or methods described herein, the upstream process begins with the inoculum stage using one or more vials of a cell bank. In some embodiments of any of the processes or methods described herein, the cell bank is a Master Cell bank (MCB) or a Working Cell Bank (WCB). In some embodiments, two vials of the cell bank are used. In some embodiments, two vials of a WCB are used. In some embodiments, the production-scale fermentation is performed using about a 1,000 L to about a 5,000 L (e.g., a 1,500-L) working volume fermenter. During the fermentation process, a continuous nutrient feed (fed-batch production) containing glucose, yeast extract, methionine, and leucine is added to maintain growth and minimize or prevent amino acid misincorporation. In some embodiments, the amino acid misincorporation is norvaline or norleucine incorporation. In some embodiments, product formation can be induced by addition of isopropyl-β-D-thiogalactopyranoside (IPTG). The harvest operations separate the cells from the fermentation broth using, e.g., centrifugation. The cells are subsequently lysed by, e.g., high pressure homogenization to release the inclusion bodies (IB). The resuspended IB can then be washed by centrifugation. In some embodiments, the resulting washed inclusion bodies (WIB) can be, e.g., refrigerated before subsequent purification operations. In some embodiments, the resulting WIB can be frozen and stored for subsequent purification operations.

In some embodiments, the downstream process can begin by suspending the WIB slurry in a solubilization buffer. The target protein can be solubilized and then oxidized, allowing the peptide chain to fold and form disulfide bonds. The oxidized product can then be purified by Dowex flow-through chromatography, acid precipitation, anion exchange chromatography, cation exchange chromatography 1, and mixed mode chromatography. Following concentration and buffer-exchange by Ultrafiltration and Diafiltration 1 (UF/DF 1), the r-met-Hu-G-CSF can be PEGylated and the reaction by-products can be removed by the cation exchange chromatography 2 step. The purified PEGylated product can be concentrated and buffer-exchanged by a second UF/DF step (UF/DF 2). Polysorbate 20 can be added to the UF/DF 2 pool, and the pH of the formulation can be adjusted to a target value (e.g., any of th exemplary target values described herein). The product can be diluted with additional diafiltration buffer to achieve a target protein concentration of 10.0 mg/mL and 0.2-μm filtered. The resulting PEGylated r-met-Hu-G-CSF is suitable as a drug substance and can be filled and stored in polyethylene terephthalate (PETG) bottles at 5±3° C.

It is noted that in this disclosure and particularly in the claims and/or paragraphs, terms such as “comprises,” “comprised,” “comprising” and the like can have the meaning attributed to it in U.S. patent law; e.g., they can mean “includes,” “included,” “including,” and the like; and that terms such as “consisting essentially of” and “consists essentially of” have the meaning ascribed to them in U.S. patent law.

These and other embodiments are disclosed or are obvious from and encompassed by, the following Detailed Description.

### DETAILED DESCRIPTION OF THE INVENTION

Provided herein are production, isolation, and purification processes or methods for PEGylated recombinant methionyl human granulocyte colony stimulating factor (r-met-Hu-G-CSF) which include an upstream process (UP) and/or a downstream processes (DSP), as described herein.

The hormone and cytokine, granulocyte colony-stimulating factor (G-CSF), is a glycoprotein that stimulates the production of white blood cells in the bone marrow and their release into the bloodstream. Granulocytes are a particular category of white blood cells that are characterized by the presence of granules in their cytoplasm. There are four types of granulocytes: neutrophils (most abundant), basophils, eosinophils, and mast cells. G-CSF is known to play a role in stimulating the survival, proliferation, differentiation, and function of neutrophil precursors and mature neutrophils. The G-CSF protein is also known as colony-stimulating factor 3 (CSF3). As used herein, “Hu-G-CSF” is a human G-CSF protein encoded by the specific gene located on chromosome 17, locus q11.2-q12 in the human genome. The G-CSF gene has 4 introns, and alternate splicing of mRNA results in two different polypeptides, differing by the presence of 3 amino acids, both of which exhibit authentic G-CSF activity.

A pharmaceutical analog of naturally occurring G-CSF is called filgrastim, a recombinant methionyl human granulocyte colony stimulating factor (r-met-Hu-G-CSF) which is a 175 amino acid protein. The protein has an amino acid sequence that is identical to the natural sequence predicted from analysis of the human genome, except for the addition of an N-terminal methionine necessary for expression in E. coli. Because filgrastim is produced in E. coli, the product is not glycosylated and thus differs from Hu-G-CSF isolated from a human cell. Filgrastim is used to treat infections and neutropenic (low white blood cells) fevers that may occur following chemotherapy, radiation poisoning, HIV/AIDS, or other unknown causes.

As used herein, a “recombinant protein” is a manipulated protein encoded by recombinant DNA. “Recombinant DNA” are DNA molecules formed by laboratory methods of molecular cloning which bring together genetic material from multiple sources, resulting in sequences that would not otherwise be found in a naturally-occurring genome. As used herein, “recombinant DNA” is DNA formed by laboratory methods which is DNA that has been cloned into a foreign expression system to support the expression of the exogenous gene.

Pegfilgrastim is a PEGylated form of filgrastim and is used for similar therapeutic purposes as filgrastim. As used herein, “PEGylate(d)” or “PEGylation” means to attach at least one PEG molecule. Pegfilgrastim persists in the body longer and has a human half-life of 15 to 80 hours, much longer than filgrastim, which is only 3 to 4 hours.

In embodiments of the invention, r-met-Hu-G-CSF is produced in genetically engineered E. coli. As used herein “E. coli” or “E. coli expression systems/strains” or “E. coli cell line(s)” include, but are not limited to, BL21 Competent E. coli, BL21(DE3) Competent E. coli, Lemo21(DE3) Competent E. coli, NEB® Express Competent E. coli (High Efficiency), NEB® Express Iq Competent E. coli (High Efficiency), NiCo21(DE3) Competent E. coli, SHuffle® Express Competent E. coli, SHuffle® T7 Competent E. coli, SHuffle® T7 Express Competent E. coli, SHuffle® T7 Express lysY Competent E. coli, T7 Express Competent E. coli (High Efficiency), T7 Express lysY Competent E. coli (High Efficiency), and T7 Express lysY/Iq Competent E. coli (High Efficiency).

### Upstream Process

The upstream process steps include but are not limited to the following: Product Fermentation, Cell Harvest, Cell Lysis, and Inclusion Body Harvest and Wash. In some embodiments, the upstream process includes an initial step of preparing a Primary Inoculum.

### Primary Inoculum

In embodiments of any of the methods or processes described herein, a primary inoculum is prepared using methods known by those of skill in the art. In some embodiments, a primary inoculum is prepared in, e.g., a shake flask. The inoculum stage provides sufficient cell mass to inoculate the production bioreactor. In some embodiments, each production culture results in a single harvest train that can be traced back to the working cell bank (WCB) vials used to initiate the primary inoculum. A WCB is prepared from master cell bank (MCB) under defined cell culture conditions. A two-tiered cell banking system consisting of a MCB and WCB is typically recommended when a cell line is to be used over many manufacturing cycles. Quality control tests, via DNA profiling techniques, are performed to confirm that the MCB and the WCB are genetically identical, as well as to ensure the WCB is free of contaminants.

In some embodiments, each 1,500-L upstream batch uses two WCB vials to inoculate seed flasks that are pooled to produce a single primary inoculum. In some embodiments, the aliquot volume from the WCB used to inoculate the seed flasks is 200 μL, 250 μL, 300 μL, 350 μL, 400 μL, 450 μL, 500 μL, 550 μL, or 600 μL. In some embodiments, the aliquot volume from the WCB used to inoculate the seed flasks is about 200 μL, to about 600 μL (e.g., about 200 μL to about 550 μL, about 200 μL to about 500 μL, about 200 μL, to about 450 μL, about 200 μL to about 400 μL, about 200 μL to about 350 μL, about 200 μL, to about 300 μL, about 200 μL, to about 250 μL, about 250 μL, to about 600 μL, about 250 μL, to about 550 μL, about 250 μL to about 500 μL, about 250 μL, to about 450 μL, about 250 μL, to about 400 μL, about 250 μL to about 350 μL, about 250 μL, to about 300 μL, about 300 μL, to about 600 μL, about 300 μL to about 550 μL, about 300 μL, to about 500 μL, about 300 μL, to about 450 μL, about 300 μL to about 400 μL, about 300 μL, to about 350 μL, about 350 μL, to about 600 μL, about 350 μL to about 550 μL, about 350 μL, to about 500 μL, about 350 μL, to about 450 μL, about 350 μL to about 400 μL, about 400 μL, to about 600 μL, about 400 μL, to about 550 μL, about 400 μL to about 500 μL, about 400 μL, to about 450 μL, about 450 μL, to about 600 μL, about 450 μL to about 550 μL, about 450 μL, to about 500 μL, about 500 μL, to about 600 μL, about 500 μL to about 550 μL, or about 550 μL, to about 600 μL.

In some embodiments of any of the methods or processes described herein, optical density (OD600) measurements are conducted to evaluate cell growth. In some embodiments, optical density measurements begin at 7, 8, 9, 10, 11, 12, 13, or 14 hours, and each hour thereafter up to 11, 12, 13, 14, 15, 16, 17, or 18 hours, respectively, until the individual flask cell masses reach an OD600 ≥2.8 (e.g., about 2.9, about 3.0, about 3.1, about 3.2, about 3.3, about 3.4, about 3.5, about 3.6, about 3.7, about 3.8, about 3.9, or about 4.0).

### Product Fermentation

Some embodiments of the product fermentation, sometimes called production-scale fermentation, are conducted to produce G-CSF. Product fermination can be performed using culture techniques and conditions known to those of skill in the art. In some embodiments, the product fermentation can be fed batch fermentation. In some embodiments, the product fermentation is batch fermentation. In some embodiments, product fermentation can be performed in a stainless-steel 1,500-L working volume bioreactor or fermenter. As used herein, “fermenter” refers to aseptic vessels used to cultivate microorganisms on a large scale. In some embodiments, product fermentation can be performed in a fermenter or bioreactor having a volume of about 100 mL to about 5,000 L (e.g., about 100 mL to about 4,500 L, about 100 mL to about 4,000 L, about 100 mL to about 3,500 L, about 100 mL to about 3,000 L, about 100 mL to about 2,500 L, about 100 mL to about 2,000 L, about 100 mL to about 1,500 L, about 100 mL to about 1,000 L, about 100 mL to about 800 mL, about 100 mL to about 600 mL, about 100 mL to about 400 mL, about 100 mL to about 200 mL, about 200 mL to about 5,000 L, about 200 mL to about 4,500 L, about 200 mL to about 4,000 L, about 200 mL to about 3,500 L, about 200 mL to about 3,000 L, about 200 mL to about 2,500 L, about 200 mL to about 2,000 L, about 200 mL to about 1,500 L, about 200 mL to about 1,000 L, about 200 mL to about 800 mL, about 200 mL to about 600 mL, about 200 mL to about 400 mL, about 400 mL to about 5,000 L, about 400 mL to about 4,500 L, about 400 mL to about 4,000 L, about 400 mL to about 3,500 L, about 400 mL to about 3,000 L, about 400 mL to about 2,500 L, about 400 mL to about 2,000 L, about 400 mL to about 1,500 L, about 400 mL to about 1,000 L, about 400 mL to about 800 mL, about 400 mL to about 600 mL, about 600 mL to about 5,000 L, about 600 mL to about 4,500 L, about 600 mL to about 4,000 L, about 600 mL to about 3,500 L, about 600 mL to about 3,000 L, about 600 mL to about 2,500 L, about 600 mL to about 2,000 L, about 600 mL to about 1,500 L, about 600 mL to about 1,000 L, about 600 mL to about 800 mL, about 800 mL to about 5,000 L, about 800 mL to about 4,500 L, about 800 mL to about 4,000 L, about 800 mL to about 3,500 L, about 800 mL to about 3,000 L, about 800 mL to about 2,500 L, about 800 mL to about 2,000 L, about 800 mL to about 1,500 L, about 800 mL to about 1,000 L, about 1,000 mL to about 5,000 L, about 1,000 mL to about 4,500 L, about 1,000 mL to about 4,000 L, about 1,000 mL to about 3,500 L, about 1,000 mL to about 3,000 L, about 1,000 mL to about 2,500 L, about 1,000 mL to about 2,000 L, about 1,000 mL to about 1,500 L, about 1,500 mL to about 5,000 L, about 1,500 mL to about 4,500 L, about 1,500 mL to about 4,000 L, about 1,500 mL to about 3,500 L, about 1,500 mL to about 3,000 L, about 1,500 mL to about 2,500 L, about 1,500 mL to about 2,000 L, about 2,000 mL to about 5,000 L, about 2,000 mL to about 4,500 L, about 2,000 mL to about 4,000 L, about 2,000 mL to about 3,500 L, about 2,000 mL to about 3,000 L, about 2,000 mL to about 2,500 L, about 2,500 mL to about 5,000 L, about 2,500 mL to about 4,500 L, about 2,500 mL to about 4,000 L, about 2,500 mL to about 3,500 L, about 2,500 mL to about 3,000 L, about 3,000 mL to about 5,000 L, about 3,000 mL to about 4,500 L, about 3,000 mL to about 4,000 L, about 3,000 mL to about 3,500 L, about 3,500 mL to about 5,000 L, about 3,500 mL to about 4,500 L, about 3,500 mL to about 4,000 L, about 4,000 mL to about 5,000 L, about 4,000 mL to about 4,500 L, or about 4,500 mL to about 5,000 L).

In some embodiments, during the fermentation process, a continuous nutrient feed (fed-batch production) containing glucose, yeast extract, methionine, and leucine is added to maintain growth and minimize or prevent amino acid misincorporation. In some embodiments, the amino acid misincorporation is norleucine incorporation. In some embodiments, product formation is induced by addition of isopropyl-β-D-thiogalactopyranoside (IPTG). As used herein “IPTG” is a molecular mimic of allolactose, a lactose metabolite that triggers transcription of the lac operon, and it is therefore used to induce E. coli protein expression where the gene is under the control of the lac operator.

In some embodiments, the product fermentation process can occur in two stages. The first stage can consist of cell mass accumulation and rapid cell growth. The second stage can consist of the product induction phase, where IPTG is added to the culture and the temperature is lowered. In some embodiments, the amount of IPTG added to the culture is 14.1, 14.2, 14.3, 14.4, 14.5, 14.6, 14.7, 14.8, 14.9 or 15.0 L of a 100 mM IPTG solution, corresponding to a broth concentration of at least 1.0 mM IPTG based on initial bioreactor volume. Throughout the product fermentation stage, the bioreactor pH, temperature, dissolved oxygen (DO), pressure, and agitation are controlled within normal operating ranges.

### Cell Harvest

The Cell Harvest step of the upstream process comprises harvest operations which separate the cells from the fermentation broth. In some embodiments, this step is conducted using centrifugation. High-level expression of many recombinant proteins in E. coli leads to the formation of highly aggregated protein commonly referred to as inclusion bodies (IBs). IBs are normally formed in the cytoplasm; however, if specific secretion vectors are used, they can form in the periplasmic space. IBs can be recovered from cell lysates by, e.g., low speed centrifugation.

Cell Lysis

The Cell Lysis step of the upstream process comprises processes whereby the cells are lysed to release IBs from the harvested cells. In some embodiments, cell lysis can be conducted using high pressure homogenization to release the IBs.

### Inclusion Body Harvest and Wash

The Inclusion Body Harvest and Wash step of the upstream process comprises processes in which the resuspended IBs are washed and the resulting washed inclusion bodies (WIB) are frozen and stored for subsequent purification operations, thereby separating the IBs from the liquid phase of the cell lysate and removing cell debris.

In some embodiments, the IBs can be washed by centrifugation. In some embodiments, the feed flow rate is maintained at 4.0 L/min, 5.0 L/min, 6.0 L/min, 7.0 L/min, 8.0 L/min, 9.0 L/min, or 10.0 L/min, or about 4.0 L/min to about 10.0 L/min (e.g., about 4.0 L/min to about 9.0 L/min, about 4.0 L/min to about 8.0 L/min, about 4.0 L/min to about 7.0 L/min, about 4.0 L/min to about 6.0 L/min, about 4.0 L/min to about 5.0 L/min, about 5.0 L/min to about 10.0 L/min, about 5.0 L/min to about 9.0 L/min, about 5.0 L/min to about 8.0 L/min, about 5.0 L/min to about 7.0 L/min, about 5.0 L/min to about 6.0 L/min, about 6.0 L/min to about 10.0 L/min, about 6.0 L/min to about 9.0 L/min, about 6.0 L/min to about 8.0 L/min, about 6.0 L/min to about 7.0 L/min, about 7.0 L/min to about 10.0 L/min, about 7.0 L/min to about 9.0 L/min, about 7.0 L/min to about 8.0 L/min, about 8.0 L/min to about 10.0 L/min, about 8.0 L/min to about 9.0 L/min, or about 9.0 L/min to about 10.0 L/min) and the centrate/centrifuge back pressure can be maintained at 45, 46, 47, 48, 49, 50, 51, 52, 53, 54, or 55 psig, or about 45 psig to about 55 psig (e.g., about 45 psig to about 54 psig, about 45 psig to about 52 psig, about 45 psig to about 50 psig, about 45 psig to about 48 psig, about 46 psig to about 55 psig, about 46 psig to about 54 psig, about 46 psig to about 52 psig, about 46 psig to about 50 psig, about 46 psig to about 48 psig, about 48 psig to about 55 psig, about 48 psig to about 54 psig, about 48 psig to about 52 psig, about 48 psig to about 50 psig, about 50 psig to about 55 psig, about 50 psig to about 54 psig, about 50 psig to about 52 psig, or about 52 psig to about 55 psig). In some embodiments, the resulting IB paste can be resuspended with purified water. In some embodiments, the resultant resuspended total pool mass can be in the range of about 1576 kg to about 1624 kg (e.g., about 1576 kg to about 1620 kg, about 1576 kg to about 1610 kg, about 1576 kg to about 1600 kg, about 1576 kg to about 1590 kg, about 1576 kg to about 1580 kg, about 1580 kg to about 1624 kg, about 1580 kg to about 1620 kg, about 1580 kg to about 1610 kg, about 1580 kg to about 1600 kg, about 1580 kg to about 1590 kg, about 1590 kg to about 1624 kg, about 1590 kg to about 1620 kg, about 1590 kg to about 1610 kg, about 1590 kg to about 1600 kg, about 1600 kg to about 1624 kg, about 1600 kg to about 1620 kg, about 1600 kg to about 1610 kg, about 1610 kg to about 1624 kg, about 1610 kg to about 1620 kg, or about 1620 kg to about 1624 kg). In some embodiments, the resultant total pool mass is about 1590 kg, about 1595 kg, about 1600 kg, about 1605 kg, about 1610 kg, about 1615 kg, or about 1620 kg.

### Downstream Process

The downstream process (DSP) steps include, but are not limited to, the following: Inclusion Body Thaw/Solubilization/Oxidation, Purification (e.g., Dowex Chromatography, Acid Precipitation/Clarification, Anion Exchange Chromatography, Cation Exchange Chromatography, and Mixed-mode Chromatography), Buffer Exchange (e.g., Ultrafiltration and Diafiltration), and PEGylation. In some embodiments, the DSP can further include Bulk Formulation and Fill.

In one embodiments, the DSP steps can include, but are not limited to, the following: Inclusion Body Thaw/Solubilization/Oxidation, Purification (e.g., Dowex Chromatography, Acid Precipitation/Clarification, Anion Exchange Chromatography, Cation Exchange Chromatography, and Mixed-mode Chromatography), Buffer Exchange (e.g., UF/DF), PEGylation, Purification of the PEGylated product (e.g., a second Cation Exchange Chromatography), Buffer Exchange into product formulation buffer (e.g., UF/DF 2), and Formulation and Fill.

### Thaw/Solubilization/Oxidation

The DSP can begin with the thaw of a specified mass of frozen WIB. The WIB Thaw/Solubilization/Oxidation step of the DSP functions to fold the product into its active conformation and form the appropriate disulfide bonds. In some embodiments, the mass of frozen WIB thawed is from a maximum of two upstream batches. The inclusion body slurry can be suspended in a solubilization buffer. The protein can be solubilized and then oxidized, allowing the peptide chain to fold and form disulfide bonds.

In some embodiments, the mass of thawed WIB containing the expressed r-met-Hu-G-CSF that is transferred into the solubilization solution is about 1188 g, about 1190 g, about 1200 g, about 1210 g, or about 1212 g, or about 1180 g to about 1220 g (e.g., about 1180 g to about 1210 g, about 1180 g to about 1200 g, about 1180 g to about 1190 g, about 1190 g to about 1220 g, about 1190 g to about 1210 g, about 1190 g to about 1200 g, about 1200 g to about 1220 g, about 1200 g to about 1210 g, or about 1210 g to about 1220 g). In some embodiments, the final buffer composition of the solubilization solution comprises about 7 g/L, about 8 g/L, about 9 g/L, about 10 g/L, about 11 g/L, or about 12 g/L Sarkosyl. In some embodiments, the solubilization solution comprises about 7.0 g/L to about 12.0 g/L (e.g., about 7.0 g/L to about 11.5 g/L, about 7.0 g/L to about 11.0 g/L, about 7.0 g/L to about 10.5 g/L, about 7.0 g/L to about 10.0 g/L, about 7.0 g/L to about 9.5 g/L, about 7.0 g/L to about 9.0 g/L, about 7.0 g/L to about 8.5 g/L, about 7.0 g/L to about 8.0 g/L, about 7.0 g/L to about 7.5 g/L, about 7.5 g/L to about 12.0 g/L, about 7.5 g/L to about 11.5 g/L, about 7.5 g/L to about 11.0 g/L, about 7.5 g/L to about 10.5 g/L, about 7.5 g/L to about 10.0 g/L, about 7.5 g/L to about 9.5 g/L, about 7.5 g/L to about 9.0 g/L, about 7.5 g/L to about 8.5 g/L, about 7.5 g/L to about 8.0 g/L, about 8.0 g/L to about 12.0 g/L, about 8.0 g/L to about 11.5 g/L, about 8.0 g/L to about 11.0 g/L, about 8.0 g/L to about 10.5 g/L, about 8.0 g/L to about 10.0 g/L, about 8.0 g/L to about 9.5 g/L, about 8.0 g/L to about 9.0 g/L, about 8.0 g/L to about 8.5 g/L, about 8.5 g/L to about 12.0 g/L, about 8.5 g/L to about 11.5 g/L, about 8.5 g/L to about 11.0 g/L, about 8.5 g/L to about 10.5 g/L, about 8.5 g/L to about 10.0 g/L, about 8.5 g/L to about 9.5 g/L, about 8.5 g/L to about 9.0 g/L, about 9.0 g/L to about 12.0 g/L, about 9.0 g/L to about 11.5 g/L, about 9.0 g/L to about 11.0 g/L, about 9.0 g/L to about 10.5 g/L, about 9.0 g/L to about 10.0 g/L, about 9.0 g/L to about 9.5 g/L, about 9.5 g/L to about 12.0 g/L, about 9.5 g/L to about 11.5 g/L, about 9.5 g/L to about 11.0 g/L, about 9.5 g/L to about 10.5 g/L, about 9.5 g/L to about 10.0 g/L, about 10.0 g/L to about 12.0 g/L, about 10.0 g/L to about 11.5 g/L, about 10.0 g/L to about 11.0 g/L, about 10.0 g/L to about 10.5 g/L, about 10.5 g/L to about 12.0 g/L, about 10.5 g/L to about 11.5 g/L, about 10.5 g/L to about 11.0 g/L, about 11.0 g/L to about 12.0 g/L, about 11.0 g/L to about 11.5 g/L, or about 11.5 g/L to about 12.0 g/L) Sarkosyl. In some embodiments, the final buffer composition of the solubilization solution comprises about 10 mM, about 15 mM, about 20 mM, or about 25 mM Tris. In some embodiments, the solubilization solution comprises about 10 mM to about 25 mM (e.g., about 10 mM to about 20 mM, about 10 mM to about 15 mM, about 15 mM to about 25 mM, about 15 mM to about 20 mM, about 20 mM to about 25 mM) Tris. In some embodiments, the final buffer composition of the solubilization solution has a pH of about 7.8, about 7.9, about 8.0, about 8.1, or about 8.2, or about 7.8 to about 8.2 (e.g., about 7.8 to about 8.1, about 7.8 to about 8.0, about 7.9 to about 8.2, about 7.9 to about 8.1, or about 8.0 to about 8.2). In some embodiments, the product concentration in the solubilization pool is about 4 g/L, about 4.5 g/L, about 5.0 g/L, or about 5.5 g/L, or about 4.0 g/L to about 5.5 g/L (e.g., about 4.0 g/L to about 5.0 g/L, about 4.0 g/L to about 4.5 g/L, about 4.5 g/L to about 5.5 g/L, about 4.5 g/L to about 5.0 g/L, or about 5.0 g/L to about 5.5 g/L). In some embodiments, the solubilization time is about 1.5 hours, about 2.0 hours, or about 2.5 hours, or about 1.5 hours to about 2.5 hours (e.g., about 1.5 hours to about 2.0 hours, or about 2.0 hours to about 2.5 hours). During the oxidation phase, the refold process is controlled at pH of about 7.8, about 7.9, about 8.0, about 8.1, or about 8.2, or about 7.8 to about 8.2 (e.g., about 7.8 to about 8.1, about 7.8 to about 8.0, about 7.9 to about 8.2, about 7.9 to about 8.1, or about 8.0 to about 8.2). In some embodiments, the refold process is controlled at temperature of about 17° C., about 18° C., about 19° C., about 20° C., about 21° C., about 22° C., or about 23° C., or about 17° C. to about 23° C. (e.g., about 17° C. to about 22° C., about 17° C. to about 21° C., about 17° C. to about 20° C., about 17° C. to about 19° C., about 18° C. to about 23° C., about 18° C. to about 22° C., about 18° C. to about 21° C., about 18° C. to about 20° C., about 19° C. to about 23° C., about 19° C. to about 22° C., about 19° C. to about 21° C., about 20° C. to about 23° C., about 20° C. to about 22° C., or about 21° C. to about 23° C.).

### Purification

In some embodiments, G-CSF can be purified by one or more of: flow-through chromatography, Acid Precipitation/Clarification, Anion Exchange Chromatography, Cation Exchange Chromatography, and Mixed Mode Chromatography. In some embodiments, purification of G-CSF incudes, but is not limited to, the following steps: Dowex flow-through chromatography, Acid Precipitation/Clarification, Anion Exchange Chromatography, Cation Exchange Chromatography, and Mixed Mode Chromatography.

### Dowex Chromatography

The purpose of the Dowex Chromatography step of the DSP is to remove detergent (e.g., sodium lauroyl sarcosinate, also refered to as Sarkosyl) from the quenched oxidation solution. In some embodiments, the resin mass can be about 22.6 kg, about 22.7 kg, about 22.8 kg, about 22.9 kg, or about 23.0 kg. In some embodiments, the resin mass can be about 20 kg to about 30 kg (e.g., about 20 kg to about 28 kg, about 20 kg to about 26 kg, about 20 kg to about 24 kg, about 20 kg to about 22 kg, about 22 kg to about 30 kg, about 22 kg to about 28 kg, about 22 kg to about 26 kg, about 22 kg to about 24 kg, about 24 kg to about 30 kg, about 24 kg to about 28 kg, about 24 kg to about 26 kg, about 26 kg to about 30 kg, about 26 kg to about 28 kg, or about 28 kg to about 30 kg). In some embodiments, the load factor measured in g Sarkosyl/L resin is about 72, about 73, about 74, about 75, about 76, about 77, or about 78. In some embodiments, the load factor, measured in g Sarkosyl/L resin is about 70 to about 80 (e.g., about 70 to about 78, about 70 to about 76, about 70 to about 74, about 70 to about 72, about 72 to about 80, about 72 to about 78, about 72 to about 76, about 72 to about 74, about 74 to about 80, about 74 to about 78, about 74 to about 76, about 76 to about 80, about 76 to about 78, or about 78 to about 80). In some embodiments, the load/wash flow rate is about 1.5 L/min, 1.6 L/min, about 1.7 L/min, about 1.8 L/min, or about 1.9 L/min. In some embodiments, the load/wash flow rate is about 1.0 L/min to about 2.0 L/min (e.g., about 1.0 L/min to about 1.8 L/min, about 1.0 L/min to about 1.6 L/min, about 1.0 L/min to about 1.4 L/min, about 1.0 L/min to about 1.2 L/min, about 1.2 L/min to about 2.0 L/min, about 1.2 L/min to about 1.8 L/min, about 1.2 L/min, about 1.6 L/min, about 1.2 L/min to about 1.4 L/min, about 1.4 L/min to about 2.0 L/min, about 1.4 L/min to about 1.8 L/min, about 1.4 L/min to about 1.6 L/min, about 1.6 L/min to about 2.0 L/min, about 1.6 L/min to about 1.8 L/min, or about 1.8 L/min to about 2.0 L/min). In some embodiments, when product collection ends, the column volume (CV) after start of wash is about 0.9, about 1.0 or about 1.1. In some embodiments, when product collection ends, the column volume (CV) after start of wash is about 0.8 to about 2.0 (e.g., about 0.8 to about 1.8, about 0.8 to about 1.6, about 0.8 to about 1.4, about 0.8 to about 1.2, about 0.8 to about 1.0, about 1.0 to about 2.0, about 1.0 to about 1.8, about 1.0 to about 1.6, about 1.0 to about 1.4, about 1.0 to about 1.2, about 1.2 to about 2.0, about 1.2 to about 1.8, about 1.2 to about 1.6, about 1.2 to about 1.4, about 1.4 to about 2.0, about 1.4 to about 1.8, about 1.4 to about 1.6, about 1.6 to about 2.0, about 1.6 to about 1.8, or about 1.8 to about 2.0). In embodiments of the invention, the anion exchange resin is Dowex AG1, Dowex AG2, Dowex AG4, BioRex5, or Dowex AG MP.

### Acid Precipitation/Clarification

The purpose of the Acid Precipitation/Clarification step of the DSP is to decrease the levels of host-cell derived impurities from the product stream in preparation for anion exchange chromatography. In some embodiments, the Dowex pool is titrated to a pH of about 4.3, about 4.4, about 4.5, about 4.6, or about 4.7 with 1.0 M acetic acid. In some embodiments, the Dowex pool is titrated to a pH of about 3.8 to about 5.0 (e.g., about 3.8 to about 4.8, about 3.8 to about 4.6, about 3.8 to about 4.4, about 3.8 to about 4.2, about 3.8 to about 4.0, about 4.0 to about 5.0, about 4.0 to about 4.8, about 4.0 to about 4.6, about 4.0 to about 4.4, about 4.0 to about 4.2, about 4.2 to about 5.0, about 4.2 to about 4.8, about 4.2 to about 4.6, about 4.2 to about 4.4, about 4.4 to about 5.0, about 4.4 to about 4.8, about 4.4 to about 4.6, about 4.6 to about 5.0, about 4.6 to about 4.8, or about 4.8 to about 5.0). In some embodiments, the acidified pool is mixed for 15, 16, 17, 18, 19, 20, 21, 22, 23, 24, or 25 minutes at 17, 18, 19, 20, 21, 22 or 23° C. In some embodiments, the acidified pool is mixed for about 15 mintes to about 30 minutes (e.g., about 15 minutes to about 25 minutes, about 15 minutes to about 20 minutes, about 20 minutes to about 30 minutes, about 20 minutes to about 25 minutes, or about 25 minutes to about 30 minutes) at a temperature of about 15° C. to about 25° C. (e.g., about 15° C. to about 24° C., about 15° C. to about 23° C., about 15° C. to about 22° C., about 15° C. to about 21° C., about 15° C. to about 20° C., about 15° C. to about 19° C., about 15° C. to about 18° C., about 15° C. to about 17° C., about 16° C. to about 25° C., about 16° C. to about 24° C., about 16° C. to about 23° C., about 16° C. to about 22° C., about 16° C. to about 21° C., about 16° C. to about 20° C., about 16° C. to about 19° C., about 16° C. to about 18° C., about 17° C. to about 25° C., about 17° C. to about 24° C., about 17° C. to about 23° C., about 17° C. to about 22° C., about 17° C. to about 21° C., about 17° C. to about 20° C., about 17° C. to about 19° C., about 18° C. to about 25° C., about 18° C. to about 24° C., about 18° C. to about 23° C., about 18° C. to about 22° C., about 18° C. to about 21° C., about 18° C. to about 20° C., about 19° C. to about 25° C., about 19° C. to about 24° C., about 19° C. to about 23° C., about 19° C. to about 22° C., about 19° C. to about 21° C., about 20° C. to about 25° C., about 20° C. to about 24° C., about 20° C. to about 23° C., about 20° C. to about 22° C., about 21° C. to about 25° C., about 21° C. to about 24° C., about 21° C. to about 23° C., about 22° C. to about 25° C., about 22° C. to about 24° C., or about 23° C. to about 25° C.).

### Anion Exchange Chromatography

The Anion Exchange Chromatography step of the DSP is used to further purify the r-met-Hu-G-CSF present in the clarified pool by reducing impurities such as host-cell proteins (HCP), DNA, and product-related variants.

In some embodiments, the chromatography system can be a GE Healthcare BioProcess skid. In some embodiments, the anion exchange resin can be TSKgelDEAE-5PW or Toyopearl DEAE-650M. In some embodiments, the load factor at this step is about 3.6, about 3.8, about 4.0, about 4.2, about 4.4, about 4.6, about 4.8, about 5.0, about 5.2, about 5.4, about 5.6, about 5.8, about 6.0, about 6.2, about 6.4, about 6.6, about 6.8, about 7.0, about 7.2, about 7.4, about 7.6, about 7.8, or about 8.0 g/L resin. In some embodiments, the load factor at this step is about 3.6 g/L resin to about 8.0 g/L resin (e.g., about 3.6 g/L resin to about 7.8 g/L resin, about 3.6 g/L resin to about 7.6 g/L resin, about 3.6 g/L resin to about 7.4 g/L resin, about 3.6 g/L resin to about 7.2 g/L resin, about 3.6 g/L resin to about 7.0 g/L resin, about 3.6 g/L resin to about 6.8 g/L resin, about 3.6 g/L resin to about 6.6 g/L resin, about 3.6 g/L resin to about 6.4 g/L resin, about 3.6 g/L reisn to about 6.2 g/L resin, about 3.6 g/L resin to about 6.0 g/L resin, about 3.6 g/L resin to about 5.8 g/L resin, about 3.6 g/L resin to about 5.6 g/L resin, about 3.6 g/L resin to about 5.4 g/L resin, about 3.6 g/L resin to about 5.2 g/L resin, about 3.6 g/L resin to about 5.0 g/L resin, about 3.6 g/L resin to about 4.8 g/L resin, about 3.6 g/L resin to about 4.6 g/L resin, about 3.6 g/L resin to about 4.4 g/L resin, about 3.6 g/L resin to about 4.2 g/L resin, about 3.6 g/L resin to about 4.0 g/L resin, about 3.6 g/L resin to about 3.8 g/L resin, about 4.0 g/L resin to about 8.0 g/L resin, about 4.0 g/L resin to about 7.8 g/L resin, about 4.0 g/L resin to about 7.6 g/L resin, about 4.0 g/L resin to about 7.4 g/L resin, about 4.0 g/L resin to about 7.2 g/L resin, about 4.0 g/L resin to about 7.0 g/L resin, about 4.0 g/L resin to about 6.8 g/L resin, about 4.0 g/L resin to about 6.6 g/L resin, about 4.0 g/L resin to about 6.4 g/L resin, about 4.0 g/L reisn to about 6.2 g/L resin, about 4.0 g/L resin to about 6.0 g/L resin, about 4.0 g/L resin to about 5.8 g/L resin, about 4.0 g/L resin to about 5.6 g/L resin, about 4.0 g/L resin to about 5.4 g/L resin, about 4.0 g/L resin to about 5.2 g/L resin, about 4.0 g/L resin to about 5.0 g/L resin, about 4.0 g/L resin to about 4.8 g/L resin, about 4.0 g/L resin to about 4.6 g/L resin, about 4.0 g/L resin to about 4.4 g/L resin, about 4.0 g/L resin to about 4.2 g/L resin, about 5.0 g/L resin to about 8.0 g/L resin, about 5.0 g/L resin to about 7.8 g/L resin, about 5.0 g/L resin to about 7.6 g/L resin, about 5.0 g/L resin to about 7.4 g/L resin, about 5.0 g/L resin to about 7.2 g/L resin, about 5.0 g/L resin to about 7.0 g/L resin, about 5.0 g/L resin to about 6.8 g/L resin, about 5.0 g/L resin to about 6.6 g/L resin, about 5.0 g/L resin to about 6.4 g/L resin, about 5.0 g/L reisn to about 6.2 g/L resin, about 5.0 g/L resin to about 6.0 g/L resin, about 5.0 g/L resin to about 5.8 g/L resin, about 5.0 g/L resin to about 5.6 g/L resin, about 5.0 g/L resin to about 5.4 g/L resin, about 5.0 g/L resin to about 5.2 g/L resin, about 6.0 g/L resin to about 8.0 g/L resin, about 6.0 g/L resin to about 7.8 g/L resin, about 6.0 g/L resin to about 7.6 g/L resin, about 6.0 g/L resin to about 7.4 g/L resin, about 6.0 g/L resin to about 7.2 g/L resin, about 6.0 g/L resin to about 7.0 g/L resin, about 6.0 g/L resin to about 6.8 g/L resin, about 6.0 g/L resin to about 6.6 g/L resin, about 6.0 g/L resin to about 6.4 g/L resin, about 6.0 g/L reisn to about 6.2 g/L resin, about 7.0 g/L resin to about 8.0 g/L resin, about 7.0 g/L resin to about 7.8 g/L resin, about 7.0 g/L resin to about 7.6 g/L resin, about 7.0 g/L resin to about 7.4 g/L resin, or about 7.0 g/L resin to about 7.2 g/L resin).

In some embodiments, the process pH (elution) is about 6.8, about 6.9, about 7.0, about 7.1, or about 7.2, or about 6.8 to about 7.2, about 6.8 to about 7.1, about 6.8 to about 7.0, about 6.9 to about 7.2, about 6.9 to about 7.1, or about 7.0 to about 7.2. In some embodiments, the temperature at this step is controlled in the range of 3-13° C., 4-12° C., or 5-11° C. In some embodiments, the product is eluted over about 5.9 CV, about 6.0 CV, or about 6.1 CV. In embodiments of the invention, the main product eluate pool collection stops when the UV₂₈₀ value reaches 65, 66, 67, 68, 69, 70, 71, 72, 73, 74, or 75% of the peak maximum UV₂₈₀ value.

### Cation Exchange Chromatography

In some embodiments, a Cation Exchange Chromatography step of the DSP further purifies r-met-Hu-G-CSF by reducing HCP, DNA, and product-related variants present in the Anion Exchange Chromatography pool. In some embodiments where more than one Cation Exchange Chromatography step is used, they may be distinguished by suffix numeral (e.g., Cation Exchange Chromatography 1 and Cation Exchange Chromatography 2).

In some embodiments, the chromatography system is a GE Healthcare BioProcess skid. In some embodiments, the cation exchange resin is SP Sepharose Fast Flow or CM Sepharose Fast Flow. In some embodiments, the load factor at this step is about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, or about 5.9 g/L resin. In some embodiments, the load factor at this step is about 2.2 g/L resin to about 6.0 g/L resin (e.g., about 2.2 g/L resin to about 5.8 g/L resin, about 2.2 g/L resin to about 5.6 g/L resin, about 2.2 g/L resin to about 5.4 g/l resin, about 2.2 g/L resin to about 5.2 g/L resin, about 2.2 g/L resin to about 5.0 g/L resin, about 2.2 g/L resin to about 4.8 g/L resin, about 2.2 g/L resin to about 4.6 g/L resin, about 2.2 g/L resin to about 4.4 g/L resin, about 2.2 g/L resin to about 4.2 g/L resin, about 2.2 g/L resin to about 4.0 g/L resin, about 2.2 g/L resin to about 3.8 g/L resin, about 2.2 g/L resin to about 3.6 g/L resin, about 2.2 g/L resin to about 3.4 g/L resin, about 2.2 g/L resin to about 3.2 g/L resin, about 2.2 g/L resin to about 3.0 g/L resin, about 2.2 g/L resin to about 2.8 g/L resin, about 2.2 g/L resin to about 2.6 g/L resin, about 2.2 g/L resin to about 2.4 g/L resin, about 3.0 g/L resin to about 6.0 g/L resin, about 3.0 g/L resin to about 5.8 g/L resin, about 3.0 g/L resin to about 5.6 g/L resin, about 3.0 g/L resin to about 5.4 g/l resin, about 3.0 g/L resin to about 5.2 g/L resin, about 3.0 g/L resin to about 5.0 g/L resin, about 3.0 g/L resin to about 4.8 g/L resin, about 3.0 g/L resin to about 4.6 g/L resin, about 3.0 g/L resin to about 4.4 g/L resin, about 3.0 g/L resin to about 4.2 g/L resin, about 3.0 g/L resin to about 4.0 g/L resin, about 3.0 g/L resin to about 3.8 g/L resin, about 3.0 g/L resin to about 3.6 g/L resin, about 3.0 g/L resin to about 3.4 g/L resin, about 3.0 g/L resin to about 3.2 g/L resin, about 4.0 g/L resin to about 6.0 g/L resin, about 4.0 g/L resin to about 5.8 g/L resin, about 4.0 g/L resin to about 5.6 g/L resin, about 4.0 g/L resin to about 5.4 g/l resin, about 4.0 g/L resin to about 5.2 g/L resin, about 4.0 g/L resin to about 5.0 g/L resin, about 4.0 g/L resin to about 4.8 g/L resin, about 4.0 g/L resin to about 4.6 g/L resin, about 4.0 g/L resin to about 4.4 g/L resin, about 4.0 g/L resin to about 4.2 g/L resin, about 5.0 g/L resin to about 6.0 g/L resin, about 5.0 g/L resin to about 5.8 g/L resin, about 5.0 g/L resin to about 5.6 g/L resin, about 5.0 g/L resin to about 5.4 g/l resin, about 5.0 g/L resin to about 5.2 g/L resin).

In some embodiments, the process pH (elution) is about 5.2, about 5.4, or about 5.6, or about 5.2 to about 5.6, about 5.2 to about 5.4, or about 5.4 to about 5.6. In some embodiments, the temperature at this step is controlled in the range of 3-13° C., 4-12° C., or 5-11° C. In some embodiments, the product is eluted over about 12.4 CV, about 12.5 CV, or about 12.6 CV. In embodiments of the invention, the main product eluate pool collection stops when the UV₂₈₀ value reaches 60, 65, 70, 75, or 80% of the peak maximum UV₂₈₀ value.

Mixed Mode Chromatography

In some embodiments, a Mixed Mode Chromatography step of the DSP is used to purify the r-met-Hu-G-CSF by decreasing product related variants. In some embodiments, the Mixed Mode Chromatography step decreases product related variants present in the Cation Exchange Chromatography 1 in-process pool.

In some embodiments, the mixed mode material is PPA Hypercel, HEA Hypercel, MEP Hypercel, Capto MIVIC, or Capto Adhere. In some embodiments, the load factor at this step is about 5.1, about 5.2, about 5.5, about 5.9, about 6.3, about 6.7, about 7.1, about 7.5, about 7.9, about 8.1, about 8.2, about 8.3, about 8.9, about 9.1, about 9.2, about 9.4, about 9.7, about 10.2, about 10.5, about 11.3, about 11.7, about 12.7, about 13.1, or about 13.3 g/L resin. In some embodiments, the load factor at this step is about 5.0 g/L resin to about 13.5 g/L resin (e.g., about 5.0 g/L resin to about 13.0 g/L resin, about 5.0 g/L resin to about 12.5 g/L resin, about 5.0 g/L resin to about 12.0 g/L resin, about 5.0 g/L resin to about 11.5 g/L resin, about 5.0 g/L resin to about 11.0 g/L resin, about 5.0 g/L resin to about 10.5 g/L resin, about 5.0 g/L resin to about 10.0 g/L resin, about 5.0 g/L resin to about 9.5 g/L resin, about 5.0 g/L resin to about 9.0 g/L resin, about 5.0 g/L resin to about 8.5 g/L resin, about 5.0 g/L resin to about 8.0 g/L resin, about 5.0 g/L resin to about 7.5 g/L resin, about 5.0 g/L resin to about 7.0 g/L resin, about 5.0 g/L resin to about 6.5 g/L resin, about 5.0 g/L resin to about 6.0 g/L resin, about 5.0 g/L resin to about 5.5 g/L resin, about 6.0 g/L resin to about 13.5 g/L resin, about 6.0 g/L resin to about 13.0 g/L resin, about 6.0 g/L resin to about 12.5 g/L resin, about 6.0 g/L resin to about 12.0 g/L resin, about 6.0 g/L resin to about 11.5 g/L resin, about 6.0 g/L resin to about 11.0 g/L resin, about 6.0 g/L resin to about 10.5 g/L resin, about 6.0 g/L resin to about 10.0 g/L resin, about 6.0 g/L resin to about 9.5 g/L resin, about 6.0 g/L resin to about 9.0 g/L resin, about 6.0 g/L resin to about 8.5 g/L resin, about 6.0 g/L resin to about 8.0 g/L resin, about 6.0 g/L resin to about 7.5 g/L resin, about 6.0 g/L resin to about 7.0 g/L resin, about 6.0 g/L resin to about 6.5 g/L resin, about 7.0 g/L resin to about 13.5 g/L resin, about 7.0 g/L resin to about 13.0 g/L resin, about 7.0 g/L resin to about 12.5 g/L resin, about 7.0 g/L resin to about 12.0 g/L resin, about 7.0 g/L resin to about 11.5 g/L resin, about 7.0 g/L resin to about 11.0 g/L resin, about 7.0 g/L resin to about 10.5 g/L resin, about 7.0 g/L resin to about 10.0 g/L resin, about 7.0 g/L resin to about 9.5 g/L resin, about 7.0 g/L resin to about 9.0 g/L resin, about 7.0 g/L resin to about 8.5 g/L resin, about 7.0 g/L resin to about 8.0 g/L resin, about 7.0 g/L resin to about 7.5 g/L resin, about 8.0 g/L resin to about 13.5 g/L resin, about 8.0 g/L resin to about 13.0 g/L resin, about 8.0 g/L resin to about 12.5 g/L resin, about 8.0 g/L resin to about 12.0 g/L resin, about 8.0 g/L resin to about 11.5 g/L resin, about 8.0 g/L resin to about 11.0 g/L resin, about 8.0 g/L resin to about 10.5 g/L resin, about 8.0 g/L resin to about 10.0 g/L resin, about 8.0 g/L resin to about 9.5 g/L resin, about 8.0 g/L resin to about 9.0 g/L resin, about 8.0 g/L resin to about 8.5 g/L resin, about 9.0 g/L resin to about 13.5 g/L resin, about 9.0 g/L resin to about 13.0 g/L resin, about 9.0 g/L resin to about 12.5 g/L resin, about 9.0 g/L resin to about 12.0 g/L resin, about 9.0 g/L resin to about 11.5 g/L resin, about 9.0 g/L resin to about 11.0 g/L resin, about 9.0 g/L resin to about 10.5 g/L resin, about 9.0 g/L resin to about 10.0 g/L resin, about 9.0 g/L resin to about 9.5 g/L resin, about 10.0 g/L resin to about 13.5 g/L resin, about 10.0 g/L resin to about 13.0 g/L resin, about 10.0 g/L resin to about 12.5 g/L resin, about 10.0 g/L resin to about 12.0 g/L resin, about 10.0 g/L resin to about 11.5 g/L resin, about 10.0 g/L resin to about 11.0 g/L resin, about 10.0 g/L resin to about 10.5 g/L resin, about 11.0 g/L resin to about 13.5 g/L resin, about 11.0 g/L resin to about 13.0 g/L resin, about 11.0 g/L resin to about 12.5 g/L resin, about 11.0 g/L resin to about 12.0 g/L resin, about 11.0 g/L resin to about 11.5 g/L resin, about 12.0 g/L resin to about 13.5 g/L resin, about 12.0 g/L resin to about 13.0 g/L resin, about 12.0 g/L resin to about 12.5 g/L resin, or about 13.0 g/L resin to about 13.5 g/L resin).

In some embodiments, the process pH (elution) is about 4.8, about 4.9, about 5.0, about 5.1, or about 5.2, or about 4.8 to about 5.1, about 4.8 to about 5.0, about 4.9 to about 5.2, about 4.9 to about 5.1, or about 5.0 to about 5.2. In embodiments of the invention, the main product eluate pool collection stops when the UV₂₈₀ value reaches 55, 56, 57, 58, 59, or 60% of the peak maximum UV₂₈₀ value.

### Buffer Exchange

In some embodiments of the invention, G-CSF is exchanged from one buffer into another. In some embodiments, the buffer exchange is by Ultrafiltration and Diafiltration (UF/DF). In some embodiments, the DSP includes more than one buffer exchange. In some embodiments, the PEGylated G-CSF can be concentrated and formulated. In some embodiments, following buffer exchange, the product can be passed through a 0.2-μm filter, and the resulting PEGylated drug substance is stored in PETG bottles at 5±3° C.

In some embodiments that include more than one buffer exchange step, the steps may be distinguished by suffix numeral (e.g. UF/DF 1 and UF/DF 2). In some embodiments, the DSP includes a first buffer exchange after purification and before PEGylation. In some embodiments, the DSP includes a second buffer exchange to exchange the product into a formulation buffer.

In some embodiments, the Ultrafiltration and Diafiltration 1 step of the DSP concentrates and buffer-exchanges a purified pool (e.g., a Mixed Mode Chromatography pool) in preparation for the PEGylation reaction. In some embodiments, the regenerated cellulose membrane has an area of about 5.5, about 6.5, or about 7.5 m², or about 5.5 m² to about 7.5 m², about 5.5 m² to about 6.5 m², or about 6.5 m² to about 7.5 m², and nominal molecular weight cutoff of about 3, about 4, about 5, or about 6 kDa, or about 3 kDa to about 6 kDa, about 3 kDa to about 5 kDa, about 3 kDa to about 4 kDa, about 4 kDa to about 6 kDa, about 4 kDa to about 5 kDa, or about 5 kDa to about 6 kDa. In embodiments of the invention, the target product concentration is 4.5 g/L, 5.0 g/L, or 5.5 g/L, or about 4.5 g/L to about 5.5 g/L, about 4.5 g/l to about 5.0 g/L, or about 5.0 g/L to about 5.5 g/L. In some embodiments, the retentate is buffer exchanged with about 4.5, about 5.0, about 5.5, about 6.0, or about 6.5 diavolumes of diafiltration buffer, or about 4.5 to about 6.5 diavolumes of diafiltration buffer, about 4.5 to about 5.5 diavolumes of diafiltration buffer, or about 5.5 to about 6.5 diavolumes of diafiltration buffer.

In some embodiments, the Ultrafiltration and Diafiltration 2 step of the DSP is used to exchange purified PEGylated product into final formulation buffer. In some embodiments, the regenerated cellulose membrane has an area of about 4.5, about 5.0, about 5.5, about 6.5, or about 7.5 m², or about 4.5 to about 7.5 m², about 4.5 to about 7.0 m², about 4.5 to about 6.5 m², about 4.5 to about 6.0 m², about 4.5 to about 5.5 m², about 4.5 to about 5.0 m², about 5.0 to about 7.5 m², about 5.0 to about 7.0 m², about 5.0 to about 6.5 m², about 5.0 to about 6.0 m², about 5.0 to about 5.5 m², about 5.5 to about 7.5 m², about 5.5 to about 7.0 m², about 5.5 to about 6.5 m², about 5.5 to about 6.0 m², about 6.0 to about 7.5 m², about 6.0 to about 7.0 m², about 6.0 to about 6.5 m², about 6.5 to about 7.5 m², about 6.5 to about 7.0 m², or about 7.0 to about 7.5 m²) and nominal molecular weight cutoff of about 3, about 4, about 5, or about 6 kDa, or about 3 kDa to about 6 kDa, about 3 kDa to about 5 kDa, about 3 kDa to about 4 kDa, about 4 kDa to about 6 kDa, about 4 kDa to about 5 kDa, or about 5 kDa to about 6 kDa. In embodiments of the invention, the target product concentration is about 11.7 g/L, about 11.8 g/L, about 11.9 g/L, about 12.0 g/L, about 12.1 g/L, about 12.2 g/L, or about 12.3 g/L, or about 11.0 g/L to about 12.5 g/L, about 11.0 g/L to about 12.0 g/L, about 11.0 g/L to about 11.5 g/L, about 11.5 g/L to about 12.5 g/L, about 11.5 g/L to about 12.0 g/L, or about 12.0 g/L to about 12.5 g/L. In further embodiments of the invention, the retentate is buffer exchanged with about 4.5, about 5.0, about 5.5, about 6.0, or about 6.5 diavolumes of diafiltration buffer, or about 4.5 to about 6.5 diavolumes of diafiltration buffer, about 4.5 to about 5.5 diavolumes of diafiltration buffer, or about 5.5 to about 6.5 diavolumes of diafiltration buffer.

### PEGylation

Following putification, the expressed product can be PEGylated. PEGylation can be conducted using techniques known to those of skill in the art. In some embodiments, the DSP includes concentration and buffer-exchange by UF/DF prior to PEGylation.

In some embodiments, in the PEGylation step of the DSP, an aldehyde-modified PEG molecule (mPEG-aldehyde) is covalently attached to the N-terminus of the r-met-Hu-G-CSF protein. In some embodiments, the protein is coupled with PEG using a ratio of about 4.7, about 4.8, about 4.9, about 5.0, about 5.1, about 5.2, or about 5.3 grams, or about 4.7 grams to about 5.3 grams, about 4.7 grams to about 5.2 grams, about 4.7 grams to about 5.1 grams, about 4.7 grams to about 5.0 grams, about 4.7 grams to about 4.9 grams, about 4.8 grams to about 5.3 grams, about 4.8 grams to about 5.2 grams, about 4.8 grams to about 5.1 grams, about 4.8 grams to about 5.0 grams, about 4.9 grams to about 5.3 grams, about 4.9 grams to about 5.2 grams, about 4.9 grams to about 5.1 grams, about 5.0 grams to about 5.3 grams, about 5.0 grams to about 5.2 grams, or about 5.1 grams to about 5.3 grams, of mPEG-aldehyde per gram of protein. In some embodiments of the invention, the reaction pH is about 4.8, about 4.9, about 5.0, about 5.1, or about 5.2, or about 4.8 to about 5.2, about 4.8 to about 5.0, or about 5.0 to about 5.2. In some embodiments, the reaction time is about 3.75, about 4.00, or about 4.25 hrs, or about 3.5 hours to about 4.5 hours, about 3.5 hours to about 4.0 hours, or about 4.0 hours to about 4.5 hours, and the reaction temperature is about 17° C., about 18° C., about 19° C., about 20° C., about 21° C., about 22° C., or about 23° C., or about 17° C. to about 23° C., about 17° C. to about 22° C., about 17° C. to about 21° C., about 17° C. to about 20° C., about 17° C. to about 19° C., about 18° C. to about 23° C., about 18° C. to about 22° C., about 18° C. to about 21° C., about 18° C. to about 20° C., about 19° C. to about 23° C., about 19° C. to about 22° C., about 19° C. to about 21° C., about 20° C. to about 23° C., about 20° C. to about 22° C., or about 21° C. to about 23° C.

In some embodiments, the PEGylation reaction by-products can be removed using a second Cation Exchange Chromatography step (Cation Exchange Chromatography 2). In the Cation Exchange Chromatography 2 step of the DSP, the PEGylated protein is purified by removing PEGylation variants present in the PEGylation pool. In some embodiments, the load factor at this step is about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, or about 4.4 g/L resin. In some embodiments, the load factor at this step is about 1.5 g/L resin to about 4.5 g/L resin (e.g., about 1.5 g/L resin to about 4.0 g/L resin, about 1.5 g/L resin to about 3.5 g/L resin, about 1.5 g/L resin to about 3.0 g/L resin, about 1.5 g/L resin to about 2.5 g/L resin, about 1.5 g/L resin to about 2.0 g/L resin, about 2.0 g/L resin to about 4.5 g/L resin, about 2.0 g/L resin to about 4.0 g/L resin, about 2.0 g/L resin to about 3.5 g/L resin, about 2.0 g/L resin to about 3.0 g/L resin, about 2.0 g/L resin to about 2.5 g/L resin, about 2.5 g/L resin to about 4.5 g/L resin, about 2.5 g/L resin to about 4.0 g/L resin, about 2.5 g/L resin to about 3.5 g/L resin, about 2.5 g/L resin to about 3.0 g/L resin, about 3.0 g/L resin to about 4.5 g/L resin, about 3.0 g/L resin to about 4.0 g/L resin, about 3.0 g/L resin to about 3.5 g/L resin, about 3.5 g/L resin to about 4.5 g/L resin, about 3.5 g/L resin to about 4.0 g/L resin, or about 4.0 g/L resin to about 4.5 g/L resin). In embodiments of the invention, the process pH (elution) is about 5.2, about 5.4, or about 5.6, or about 5.2 to about 5.6, about 5.2 to about 5.4, or about 5.4 to about 5.6. In some embodiments, the gradient length, in CVs, is about 8.3, about 8.4, or about 8.5. In embodiments of the invention, the main product eluate pool collection stops when the UV₂₈₀ value reaches 30, 35, or 40% of the peak maximum UV₂₈₀ value.

### Bulk Formulation and Fill

The Formulation and Fill step of the DSP is to ensure that the drug substance expressed product achieves the specified concentration and is filled into clean certified sterile containers. In some embodiments, the formulation is adjusted so that the r-met-Hu-G-CSF is present at about 10 mg/mL. In some embodiments, the drug product is filled into a sterile syringe.

### Buffers

Buffers are prepared in a batch process where components are dispensed into a defined quantity of water and mixed to homogeneity, 0.2-μm filtered, and stored according to approved site procedures. Product-contacting buffers and solutions for process steps prior to mixed mode chromatography are prepared using purified water (PW). Product-contacting buffers and solutions for process steps from Mixed Mode Chromatography through Formulation/Fill can be prepared using water for injection (WFI). Each buffer has defined composition limits and can be controlled for ingredient weight and pH per buffer batch records. Any buffer not meeting its release criteria can be discarded. Routine processing can be performed at controlled room temperature (17° C.-23° C.). Product pool bioburden and endotoxin samples can be taken at the end of the pool hold duration prior to 0.2-μm filtration.

Any patents, patent publications, and applications, and all documents cited therein or during their prosecution (“appin cited documents”) and all documents cited or referenced in the appin cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appin cited documents) are incorporated herein by reference to the same extent as if each individual document was specifically and individually indicated to be incorporated by reference.

The present invention will be further illustrated in the following Examples which are given for illustration purposes only and are not intended to limit the invention in any way.

### EXAMPLES

### Example 1: Shake Flask Primary Inoculum

The inoculum step of the UP process uses kanamycin in the medium to maintain selective pressure and does not include product induction. The primary inoculum is a shake flask culture, initiated with two WCB vials. The vials are removed from −70° C. storage, thawed, and at least 200 μL of each vial is inoculated into each of the six 4-L shake flasks, each of which contains 1.2 L of medium containing 20.0 g/L Select APS LB Broth Base and 0.05 g/L of kanamycin sulfate. The flasks are incubated in a shaking incubator at 230-270 revolutions per minute (rpm) and 30° C. Incubator agitation and temperature are monitored and controlled.

Optical density (OD600) measurements are taken until the individual flask cell masses reach an OD600 ≥2.8. The entire contents of the six shake flasks are then pooled in a biosafety cabinet into a pressure can and tested to verify the pooled cell mass reached an OD600 of ≥2.8. The pressure can can be immediately transported by personnel to the production bioreactor. The pooled contents are tested for host cell purity. Exemplary process parameters for the Shake Flask Primary Inoculum Process are indicated below in Table 1.

TABLE 1

| | Unit of | Normal Operating |
|---|---|---|
| Process Parameter | Measure | Range |
| Medium quantity | kg | 1.19-1.21 |
| Agitation | rpm | 230-270 |
| Temperature | ° C. | 28-32 |

### Example 2: Production Fermentation

The production (batch) medium is prepared in the fermentor at a target volume of 1,100 L. The medium comprises yeast extract and glycerol, which are heat sterilized in the fermentor, as well as antifoam, kanamycin, and trace elements that are 0.2-μm filtered into the fermentor as soon as it has cooled down to ambient temperature. This medium is batched and sterilized in the fermentor. The production fermentor is inoculated by connecting the primary inoculum pressure can to the fermentor via a steam sterilized transfer line. The pressure can contents are transferred to the production fermentor via a pressurization of the can with filtered process air.

During the growth phase, the production culture is controlled at 36.5-37.5° C. and pH 6.6-7.0. Phosphoric acid and ammonium hydroxide are used to maintain pH control. At 6.5 hours post-inoculation (or immediately following a DO spike due to carbon limitation), a time-based nutrient feed medium addition with 9 individual feed stages at specific flow rates ranging from 20 kg/hr to 35 kg/hr for durations of 30 to 60 minutes begins. The duration of the last feed stage is through the completion of fermentation.

For the nutrient feed medium preparation, a glucose solution is prepared in a feed tank and heat sterilized. After this solution has cooled to ambient temperature, yeast extract, magnesium sulfate, ammonium sulfate, citric acid, and methionine and leucine supplements are transferred to the feed tank through a 0.2-μm filter.

The culture temperature is reduced to 34° C. at 6.5 hours after the start of the feed medium addition. Product formation is induced at 7 hours after the initiation of feed addition, by adding IPTG solution, corresponding to a broth concentration of 1.3 mM IPTG based on initial bioreactor volume. The IPTG solution is transferred into the bioreactor from a pressurized stainless-steel can connected through a 0.2-μm sterilizing filter. At 6 hours post-induction, the culture is chilled to 12° C. in preparation for harvest. Throughout the production fermentation stage, optical density (OD600) values are taken to monitor cell growth. In some embodiments, the production culture duration may not exceed 21 hours. In some embodiments, the total culture duration, defined as vial thaw to initiation of harvest, may not exceed 55 hours and 15 minutes. At the end of the culture, aseptic samples are taken for product concentration (titer) and IPC testing. Titers are typically >1.3 g/L. Exemplary process parameters for the Production Fermentation stage are provided in Table 2 below:

TABLE 2

| | Unit of | Normal Operating |
|---|---|---|
| Process Parameter | Measure | Range |
| Agitation | rpm | 50-190 |
| pH | N/A | 6.6-7.0 |
| Temperature (pre-induction) | ° C. | 36.5-37.5 |
| Temperature (at induction) | ° C. | 33.5-34.5 |
| Dissolved oxygen | % | 15-55 |
| Feed start time (duration from | hh.mm | 06:20-06:40 |
| inoculation) | | |
| Induction start (duration from | hh:mm | 06:45-07:15 |
| feed start) | | |
| Induction duration | hh:mm | 05:45-06:15 |
| Production culture duration | hh:mm | ≤21:00 |
| Maximum total culture duration | hh:mm | ≤55:15 |

### Example 3: Cell Harvest

The purpose of the Cell Harvest stage is to separate and retain cells from the liquid phase of the culture. The solid phase (cells) of the whole culture broth is separated from the liquid phase by centrifugation using, e.g., a disc-stack centrifuge. The centrifuge discharge is connected to a 2,000-L stainless-steel jacketed collection vessel via a stainless-steel transfer line. The separated solids (cell paste) accumulate in the bowl and are discharged at 20-L intervals and transferred to a collection vessel maintained at ≤15° C. During centrifugation, the feed flow rate to the centrifuge is maintained at 11.0 L/min, the bowl speed of approximately 7500 rpm, and the centrate back pressure at 50 pounds per square inch gauge (psig). Once the production culture has been processed through the centrifuge, the harvested cell paste can be diluted with purified water to 1,575 kg total pool mass. Exemplary process parameters for the Cell Harvest step are provided in Table 3 below. The cell paste can be forward-processed without interruption to the Cell Lysis step.

TABLE 3

| | Unit of | Normal Operating |
|---|---|---|
| Process Parameter | Measure | Range |
| Centrifuge feed flow rate | L/min | 9.0-12.0 |
| Centrifuge backpressure | psig | 45-55 |
| (centrate) | | |
| Discharge interval | L | 16-21 |
| Post-dilution pool mass | kg | 1550-1600 |

### Example 4: Cell Lysis

The purpose of the Cell Lysis stage is to mechanically rupture the cells to release the inclusion bodies. The cell paste feed vessel can be connected to a high-pressure homogenizer via a stainless-steel transfer line. Towards the end of the cell paste transfer, purified water is used to chase residual cell paste from the feed vessel. The homogenizer outlet is connected to a heat exchanger which feeds to a 2,000-L stainless-steel lysate collection tank. The diluted cell paste is passed through the homogenizer at least three times at a pressure of 885 bar and flow rate of 8.5 L/min. The lysate temperature can be maintained at ≤15° C. by a heat exchanger at the outlet of the homogenizer and temperature control of the collection vessel jacket. If the lysate pool mass is less than 1,590 kg, purified water can be added to the pool to achieve a mass of 1,590 kg. Exemplary process parameters for the Cell Lysis step are provided in Table 4 below. The lysate pool can be forward-processed without interruption to the Inclusion Body Harvest step.

TABLE 4

| | Unit of | Normal Operating |
|---|---|---|
| Process Parameter | Measure | Range |
| Flow rate | L/min | 6.0-10.0 |
| Break pressure | bar | 860-910 |
| Lysate temperature | ° C. | ≤15 |

### Example 5: Inclusion Body Harvest and Wash

The purpose of the Inclusion Body Harvest and Wash step of the UP is to separate the IBs from the liquid phase of the cell lysate and to remove cell debris. The lysate-containing feed vessel is connected to the disc-stack centrifuge via a stainless-steel transfer line. The centrifuge discharge is connected via a stainless-steel transfer line to the 2,000-L stainless-steel inclusion body collection tank. The separated solids accumulate in the centrifuge bowl and are discharged at 120-L intervals and transferred to a collection vessel maintained at ≤15° C. The liquid phase is sent to process waste. During centrifugation, the centrifuge feed flow rate is maintained at 8.0 L/min, the bowl speed at approximately 7500 rpm, and the centrate backpressure at 50 psig. Exemplary process parameters for the Inclusion Body Harvest are provided in Table 5 below.

TABLE 5

| | Unit of | Normal Operating |
|---|---|---|
| Process Parameter | Measure | Range |
| Centrifuge feed flow rate | L/min | 4.0-10.0 |
| Centrifuge Backpressure | psig | 45-55 |
| (centrate) | | |
| Discharge interval | L | 105-122 |

The resulting inclusion body paste can then be re-suspended with purified water to the requisite total pool mass and processed through the centrifuge a second time to further remove cell debris. The solids accumulate in the bowl and are discharged at 136-L intervals and transferred via a stainless-steel line to a single-use mixing vessel maintained at ≤20° C. The feed flow rate and centrate backpressure are maintained within the ranges disclosed. The harvested WIB are dispensed as 5-L aliquots into single-use bags. Each bag is labeled and numbered and transported by production personnel to a controlled storage area where they are placed into freezers for storage at <−60° C. for up to 24 months. Exemplary process parameters for the WIB process are provided in Table 6 below.

TABLE 6

| | Unit of | Normal Operating |
|---|---|---|
| Process Parameter | Measure | Range |
| Post-dilution pool mass | kg | 1576-1624 |
| Centrifuge feed flow rate | L/min | 4.0-10.0 |
| Centrifuge backpressure | psig | 45-55 |
| (centrate) | | |
| Centrifuge bowl speed | rpm | 7188-7788 |
| Discharge interval | L | 120-138 |
| Bag fill volume | L | <6 |
| Bag storage temperature | ° C. | ≤−60 |
| Bag storage duration | months | ≤24 |

### Example 6: WIB Thaw/Solubilization/Oxidation

In the WIB thaw phase, the frozen inclusion bodies can be thawed by static incubation at 17-23° C. for no more than 36 hours. For the Solubilization phase, the solubilization solution is prepared in a 550-L stainless-steel tank. A mass of thawed WIB containing 1188-1212 g of the expressed r-met-Hu-G-CSF (IB product mass) is transferred into the solubilization solution through a silicone tubing transfer line using a peristaltic pump and is mixed for 1.5-2.5 hours. The final buffer composition of the solubilization solution is in the range of about 8 to about 11 g/L (e.g., about 8 g/L to about 10.5 g/L, about 8 g/L to about 10 g/L, about 8 g/L to about 9.5 g/L, about 8 g/L to about 9 g/L, about 8 g/L to about 8.5 g/L, about 8.5 g/L to about 11 g/L, about 8.5 g/L to about 10.5 g/L, about 8.5 g/L to about 10 g/L, about 8.5 g/L to about 9.5 g/L, about 8.5 g/L to about 9 g/L, about 9 g/L to about 11 g/L, about 9 g/L to about 10.5 g/L, about 9 g/L to about 10 g/L, about 9 g/L to about 9.5 g/L, about 9.5 g/L to about 11 g/L, about 9.5 g/L to about 10.5 g/L, about 9.5 g/L to about 10 g/L, about 10 g/L to about 11 g/L, about 10 g/L to about 10.5 g/L, or about 10.5 g/L to about 11 g/L) Sarkosyl, about 15 to about 35 mM (e.g., about 15 mM to about 30 mM, about 15 mM to about 25 mM, about 15 mM to about 20 mM, about 20 mM to about 35 mM, about 20 mM to about 30 mM, about 20 mM to about 25 mM, about 25 mM to about 35 mM, about 25 mM to about 30 mM, or about 30 mM to about 35 mM) Tris, pH of about 7 to about 8.5 (e.g., about 7 to about 8.4, about 7 to about 8.2, about 7 to about 8, about 7 to about 7.8, about 7 to about 7.6, about 7 to about 7.4, about 7 to about 7.2, about 7.2 to about 8.5, about 7.2 to about 8.4, about 7.2 to about 8.2, about 7.2 to about 8, about 7.2 to about 7.8, about 7.2 to about 7.6, about 7.2 to about 7.4, about 7.4 to about 8.5, about 7.4 to about 8.4, about 7.4 to about 8.2, about 7.4 to about 8, about 7.4 to about 7.8, about 7.4 to about 7.6, about 7.6 to about 8.5, about 7.6 to about 8.4, about 7.6 to about 8.2, about 7.6 to about 8, about 7.6 to about 7.8, about 7.8 to about 8.5, about 7.8 to about 8.4, about 7.8 to about 8.2, about 7.8 to about 8, about 8 to about 8.5, about 8 to about 8.4, about 8 to about 8.2, about 8.2 to about 8.5, or about 8.2 to about 8.4). The target product concentration in the Solubilization pool is 4.5-5.5 g/L. The total volume of the Solubilization phase can be approximately 240 L.

After the end of the Solubilization period, the Oxidation phase begins disulfide bond formation by adding 20 mM copper sulfate stock solution to achieve a final concentration of 200 μM copper. The refold process is controlled at pH in the range of 7.8-8.2 and at temperature in the range of 17−23° C. To verify completion of the refold progress, samples are taken at regular intervals and assessed by reversed-phase high-performance liquid chromatography (RPC). The non-reduced peak area is measured in 2-hour intervals. The reaction is considered complete when the difference between non-reduced peak areas of two consecutive time points is lower than 4%. At the completion of the refold process, a stock solution of 12 mM ethylenediaminetetraacetic acid (EDTA) is added to achieve a final concentration of 600 μM EDTA in order to quench the refold reaction. In some embodiments, the maximum time from completion of the oxidation step until the subsequent Dowex step is 24 hours at 17-23° C.

### Example 7: Dowex Chromatography

The solubilization/oxidation pool is passed over the Dowex chromatography resin where the Sarkosyl is captured and the product is collected in the flow-through effluent. Prior to each downstream batch, the Dowex anion exchange resin is packed with resin mass and the packing density of Dowex resin results in a packed bed height of 40-50 cm. The packed column can be sanitized prior to use with successive washes of 4 column volumes (CV) of 1.0 M acetic acid, 5 CV of purified water (PW), and 3 CV of 1.0 M sodium hydroxide. The column can be held in 1.0 M NaOH for a minimum of 12 hours. The sanitization phase can be followed by another 5 CV PW flush.

After column sanitization, the packed column can be pre-equilibrated with 3 CV of 0.4 M Tris, 0.5 M NaCl, pH 8.0 to facilitate equilibration. The subsequent equilibration phase can be a 3 CV flush with a 20 mM Tris, pH 8.0 buffer. Before loading the column, the Solubilization/Oxidation pool can be diluted with purified water to four times the pool mass. This is accomplished by transferring the quenched Solubilization/Oxidation pool from the Solubilization/Oxidation tank through a stainless-steel transfer line into a 2,250-L stainless-steel tank using air pressure, after which 3 volumes of purified water are added to the 2,250-L tank.

The Dowex load is pumped onto the column taking into account the Sarkosyl load factor. The Sarkosyl binds to the resin as the product flows through to the collection tank. Product collection starts with the beginning of product load and ends after a 1 CV flush with 20 mM Tris, pH 8.0 after the load is complete. The eluate and flush are collected in a 2,250-L stainless-steel tank through a stainless-steel transfer line. Product pooling is controlled volumetrically, therefore monitoring of the column eluate for UV absorbance is not performed. The single-use Dowex resin is discarded and a new column is packed prior to the next run. In some embodiments, the maximum time from completion of the Dowex step until the subsequent Acidification/Clarification step is at most 8 hours at 17-23° C. Exemplary process parameters for the Dowex Chromatography step are provided in Table 7 below.

TABLE 7

| | Unit of | Normal Operating |
|---|---|---|
| Process Parameter | Measure | Range |
| Resin mass | kg | 22.6-23.0 |
| Load factor | g Sarkosyl/ | 72-78 |
| | L resin | |
| Temperature | ° C. | 17-23 |
| Load/wash flow rate | L/min | 1.5-1.9 |
| Start collect | CV after | N/A |
| | load start | |
| End collect | CV after | 0.9-1.1 |
| | start of | |
| | wash | |
| Dowex pool hold temperature | ° C. | 17-23 |
| Dowex pool hold duration | hours | ≤8 |

### Example 8: Acid Precipitation/Clarification

The purpose of this step of the DSP is to decrease the levels of host-cell derived impurities from the product stream to prepare for anion exchange chromatography. The Dowex pool is titrated to a pH in the range of 4.3-4.7 with 1.0 M acetic acid. The acidified pool is mixed for 15-25 minutes at 17-23° C. The liquid phase of the acidified pool containing product can then separated from the solid phase (waste) by centrifugation using a disc-stack centrifuge. The feed flow rate can be maintained at 14-16 L/min at a bowl speed of approximately 7500 rpm and with a centrate backpressure of 45-55 psig. The accumulated solids are discharged at a 225-L interval and discarded. The clarified centrate can be transferred from the centrifuge to a 2,250-L stainless-steel collection tank via a stainless-steel transfer line. The clarified pool can then be titrated with 1.0 N sodium hydroxide. In some embodiments, the maximum time from completion of the Clarification phase until the subsequent Anion Exchange Chromatography step is 16 hours at 17-23° C. Exemplary process parameters for the Acid Precipitation/Clarification step are provided in Table 8 below.

TABLE 8

| | Unit of | Normal Operating |
|---|---|---|
| Process Parameter | Measure | Range |
| Acidification pH | pH | 4.3-4.7 |
| Acidification temperature | ° C. | 17-23 |
| Acidification mixing speed | RPM | 100-120 |
| Acidified pool mix time | minutes | 15-25 |
| Centrifuge feed flow rate | L/min | 14-16 |
| Centrifuge backpressure (centrate) | psig | 45-55 |
| Clarified pool pH | pH | 7.8-8.2 |
| Clarified pool hold temperature | ° C. | 7-13 |
| Clarified pool hold duration | hours | ≤16 |

### Example 9: Anion Exchange Chromatography

In this step, the anion exchange resin in the chromatography system is packed in a 60-cm diameter column to a bed height of 40 cm. Prior to each use, the column can be flushed with 2 CV 0.1 M sodium hydroxide (NaOH) and sanitized with 3 CV of 1.0 N NaOH with a sanitization hold duration of at least 30 minutes at 20° C. The 1.0 N NaOH can be flushed out of the column with 2.0 CV of 0.1 N NaOH. The residence time for all chromatography phases can be 14.0 min/CV with the exception of the elution phase, which can be run at a lower flow rate, equivalent to a 22.4 min/CV residence time.

To facilitate equilibration, the sanitized column can be pre-equilibrated and then equilibrated with 3 CV of 20 mM Tris pH 8.0 buffer. From column equilibration through elution, the feed stream going to the column can be passed through a heat exchanger. The temperature can be controlled during the product-contacting phases. The clarified pool can be depth filtered and 0.2-μm filtered in-line prior to loading onto the column at a load factor in the range of about 3.6 g/L to about 8.0 g/L (e.g., about 3.6 g/L to about 7.5 g/L, about 3.6 g/L to about 7.0 g/L, about 3.6 g/L to about 6.5 g/L, about 3.6 g/L to about 6.0 g/L, about 3.6 g/L to about 5.5 g/L, about 3.6 g/L to about 5.0 g/L, about 3.6 g/L to about 4.5 g/L, about 3.6 g/L to about 4.0 g/L, about 4.0 g/L to about 8.0 g/L, about 4.0 g/L to about 7.5 g/L, about 4.0 g/L to about 7.0 g/L, about 4.0 g/L to about 6.5 g/L, about 4.0 g/L to about 6.0 g/L, about 4.0 g/L to about 5.5 g/L, about 4.0 g/L to about 5.0 g/L, about 4.0 g/L to about 4.5 g/L, about 4.5 g/L to about 8.0 g/L, about 4.5 g/L to about 7.5 g/L, about 4.5 g/L to about 7.0 g/L, about 4.5 g/L to about 6.5 g/L, about 4.5 g/L to about 6.0 g/L, about 4.5 g/L to about 5.5 g/L, about 4.5 g/L to about 5.0 g/L, about 5.0 g/L to about 8.0 g/L, about 5.0 g/L to about 7.5 g/L, about 5.0 g/L to about 7.0 g/L, about 5.0 g/L to about 6.5 g/L, about 5.0 g/L to about 6.0 g/L, about 5.0 g/L to about 5.5 g/L, about 5.5 g/L to about 8.0 g/L, about 5.5 g/L to about 7.5 g/L, about 5.5 g/L to about 7.0 g/L, about 5.5 g/L to about 6.5 g/L, about 5.5 g/L to about 6.0 g/L, about 6.0 g/L to about 8.0 g/L, about 6.0 g/L to about 7.5 g/L, about 6.0 g/L to about 7.0 g/L, about 6.0 g/L to about 6.5 g/L, about 6.5 g/L to about 8.0 g/L, about 6.5 g/L to about 7.5 g/L, about 6.5 g/L to about 7.0 g/L, about 7.0 g/L to about 8.0 g/L, about 7.0 g/L to about 7.5 g/L, or about 7.5 g/L to about 8.0 g/L) of resin. After loading, the column is washed with 3 CV of 20 mM Tris, pH 7.0 buffer. The product can then eluted using a linear gradient of increasing ionic strength, e.g., from 20 mM Tris pH 7.0 to 20 mM Tris, 100 mM NaCl, pH 7.0 over 5.9-6.1 CV.

In-process pool collection is achieved by fractionation of the product peak. The start and end of pooling is based on absorbance at 280 nm. When the UV₂₈₀ peak reaches ≥0.25 AU/cm, two 20-L fractions of eluate are collected into sterile single-use bags, prior to the collection of the main product peak fraction. The main product eluate pool collection stops when the UV₂₈₀ value reaches 65-75% of the peak maximum UV₂₈₀ value. The main product peak fraction is also collected in a sterile single-use bag. Once the peak maximum is known, the contents of 20-L fractions with initial (at the start of fraction collection) UV₂₈₀ values above 15% of peak maximum are combined with the main product fraction after the elution is complete.

The column can be cleaned with a non-denaturing ionic solution (2 CV of 2 M NaCl) followed by a sanitization with 3 CV of denaturing alkaline solution, 1 M NaOH, with a minimum sanitization duration of 30 minutes at 20° C. If the column is stored for up to two weeks (short-term storage), the column is then flushed with 3 CV of 0.1 M NaOH. If the column is stored for longer than two weeks (long-term storage), it is flushed with 3 CV of 0.4 M Tris, 0.5 M sodium chloride, pH 8.0, followed by a 3 CV flush with 1% benzyl alcohol storage solution. Exemplary process parameters for the Anion Exchange Chromatography step are provided in Table 9 below.

TABLE 9

| | Unit of | Normal Operating |
|---|---|---|
| Process Parameter | Measure | Range |
| Load factor | g/L resin | 3.6-8.0 |
| Process pH (elution) | pH | 6.8-7.2 |
| Bed height | cm | 38-42 |
| Residence time (load/wash) | Min/CV | 13.3-14.7 |
| Residence time (elution) | Min/CV | 21.3-23.5 |
| Temperature | ° C. | 4-12 |
| Gradient length | CV | 5.9-6.1 |
| Start collect | UV₂₈₀% peak | ≥15 |
| | maximum | |
| Stop collect | UV₂₈₀% peak | 65-75 |
| | maximum | |
| Pool hold temperature | ° C. | 2-8 |
| Pool hold duration | hh:mm | ≤19:29 |

### Example 10: Cation Exchange Chromatography 1

The cation exchange resin in the chromatography system is packed in a 60-cm diameter column with a bed height of 40 cm. All chromatography phases are run at the same residence time of 22.6 min/CV. Prior to each use, the column can be flushed with 2 CV 0.1 M NaOH and sanitized with 3 CV of 1.0 M sodium hydroxide. The sanitization hold duration can be at least 30 minutes at 20° C. The sanitization phase can be followed by another flush with 2 CV of 0.1 M NaOH.

To facilitate equilibration, the sanitized column can be pre-equilibrated with 1.5 CV of 1.0 M sodium acetate, 1.0 M NaCl, pH 5.4 buffer. This phase can be followed by an equilibration with 3 CV of 20 mM sodium acetate, pH 5.4 buffer. Before loading the cation exchange column, the Anion Exchange Chromatography pool can be diluted with 2 pool volumes of 20 mM acetate, pH 5.4. If required, the diluted pool can then be titrated with glacial acetic acid to pH 5.4. From column equilibration through elution, the process stream going to the column can be passed through a heat exchanger to reduce the temperature to 4-12° C. The temperature can be controlled within 4-12° C. during the product-contacting phases.

The diluted Anion Exchange Chromatography pool can be 0.2-μm filtered in-line prior to loading onto the column at a load factor in the range of about 2.3 g.L to about 5.9 g/L (e.g., about 2.3 g/L to about 5.5 g/L, about 2.3 g/L to about 5.0 g/L, about 2.3 g/L to about 4.5 g/L, about 2.3 g/L to about 4.0 g/L, about 2.3 g/L to about 3.5 g/L, about 2.3 g/L to about 3.0 g/L, about 2.3 g/L to about 2.5 g/L, about 2.5 g/L to about 5.9 g/L, about 2.5 g/L to about 5.5 g/L, about 2.5 g/L to about 5.0 g/L, about 2.5 g/L to about 4.5 g/L, about 2.5 g/L to about 4.0 g/L, about 2.5 g/L to about 3.5 g/L, about 2.5 g/L to about 3.0 g/L, about 3.0 g/L to about 5.9 g/L, about 3.0 g/L to about 5.5 g/L, about 3.0 g/L to about 5.0 g/L, about 3.0 g/L to about 4.5 g/L, about 3.0 g/L to about 4.0 g/L, about 3.0 g/L to about 3.5 g/L, about 3.5 g/L to about 5.9 g/L, about 3.5 g/L to about 5.5 g/L, about 3.5 g/L to about 5.0 g/L, about 3.5 g/L to about 4.5 g/L, about 3.5 g/L to about 4.0 g/L, about 4.0 g/L to about 5.9 g/L, about 4.0 g/L to about 5.5 g/L, about 4.0 g/L to about 5.0 g/L, about 4.0 g/L to about 4.5 g/L, about 4.5 g/L to about 5.9 g/L, about 4.5 g/L to about 5.5 g/L, about 4.5 g/L to about 5.0 g/L, about 5.0 g/L to about 5.9 g/L, about 5.0 g/L to about 5.5 g/L, or about 5.5 g/L to about 5.9 g.L) of resin. After loading, the column can be washed with 3 column volumes of 20 mM sodium acetate buffer, pH 5.2-5.6, and then eluted using a linear gradient of increasing ionic strength over 12.4-12.6 column volumes. In-process pool collection is achieved by fractionation of the product peak. All fractions are collected in sterile single use bags. The start and end of pooling is based on absorbance at 280 nm. When the UV₂₈₀ peak reaches ≥0.25 AU/cm, two 20-L fractions of eluate are collected into sterile single-use bags, prior to the collection of the main product peak fraction. The main product eluate pool collection stops when the UV₂₈₀ value reaches 60-80% of the peak maximum UV₂₈₀ value. Once the peak maximum is known, the contents of 20-L fractions with initial (at the start of fraction collection) UV₂₈₀ values above 15% of peak maximum are combined with the main product fraction after the elution is complete.

The column can be cleaned with a non-denaturing ionic solution (2 CV of 1.0 M NaCl) followed by a sanitization with 3 CV of denaturing alkaline solution, 1.0 M NaOH, with a minimum sanitization duration of 30 minutes at 20° C. The column can then be flushed with 3 CV of 0.1 M NaOH and stored until the next use. Exemplary process parameters for Cation Exchange Chromatography 1 step are provided in Table 10 below.

TABLE 10

| | Unit of | Normal Operating |
|---|---|---|
| Process Parameter | Measure | Range |
| Load factor | g/L resin | 2.3-5.9 |
| Process pH (elution) | pH | 5.2-5.6 |
| Bed height | cm | 38-42 |
| Residence time (load/ | min/CV | 21.5-23.7 |
| wash/elution) | | |
| Temperature | ° C. | 4-12 |
| Gradient Length | CV | 12.4-12.6 |
| Start collect | UV₂₈₀% peak | ≥15 |
| | maximum | |
| Stop collect | UV₂₈₀% peak | 60-80 |
| | maximum | |
| Pool hold temperature | ° C. | 2-8 |
| Pool hold duration | hh:mm | ≤14:16 |

### Example 11: Mixed Mode Chromatography

The mixed-mode resin in the chromatography system is packed in a 60-cm diameter column with a bed height of 15 cm. The residence time for all chromatography phases up to elution can be maintained at 7.0 min/CV. For the elution, post-elution cleaning and storage phase, the flow rate can be lowered to a 10.0 min/CV residence time. The Mixed Mode Chromatography step can be run at 20° C.

Prior to each use, the column can be flushed with 2 CV 0.1M NaOH and sanitized with 3 CV of 1.0 N sodium hydroxide with a sanitization duration of at least 30 minutes. This can be followed by a flush with 2 CV of 0.1N NaOH. To facilitate equilibration, the sanitized column can be pre-equilibrated with 3 CV of 100 mM acetic acid. It can then be equilibrated with 4 CV of 20 mM sodium acetate, 120 mM sodium chloride, pH 5.4 buffer.

After equilibration, the Cation Exchange Chromatography 1 pool can be 0.2-μm filtered in-line prior to loading onto the column at a load factor in the range of about 5.1 g/L to about 13.3 g/L (e.g., about 5.1 g/L to about 13.0 g/L, about 5.1 g/L to about 12.0 g/L, about 5.1 g/L to about 11.0 g/L, about 5.1 g/L to about 10.0 g/L, about 5.1 g/L to about 9.0 g/L, about 5.1 g/L to about 8.0 g/L, about 5.1 g/L to about 7.0 g/L, about 5.1 g/L to about 6.0 g/L, about 6.0 g/L to about 13.3 g/L of resin, about 6.0 g/L to about 13.0 g/L, about 6.0 g/L to about 12.0 g/L, about 6.0 g/L to about 11.0 g/L, about 6.0 g/L to about 10.0 g/L, about 6.0 g/L to about 9.0 g/L, about 6.0 g/L to about 8.0 g/L, about 6.0 g/L to about 7.0 g/L, about 7.0 g/L to about 13.3 g/L of resin, about 7.0 g/L to about 13.0 g/L, about 7.0 g/L to about 12.0 g/L, about 7.0 g/L to about 11.0 g/L, about 7.0 g/L to about 10.0 g/L, about 7.0 g/L to about 9.0 g/L, about 7.0 g/L to about 8.0 g/L, about 8.0 g/L to about 13.3 g/L of resin, about 8.0 g/L to about 13.0 g/L, about 8.0 g/L to about 12.0 g/L, about 8.0 g/L to about 11.0 g/L, about 8.0 g/L to about 10.0 g/L, about 8.0 g/L to about 9.0 g/L, about 9.0 g/L to about 13.3 g/L of resin, about 9.0 g/L to about 13.0 g/L, about 9.0 g/L to about 12.0 g/L, about 9.0 g/L to about 11.0 g/L, about 9.0 g/L to about 10.0 g/L, about 10.0 g/L to about 13.3 g/L of resin, about 10.0 g/L to about 13.0 g/L, about 10.0 g/L to about 12.0 g/L, about 10.0 g/L to about 11.0 g/L, about 11.0 g/L to about 13.3 g/L of resin, about 11.0 g/L to about 13.0 g/L, about 11.0 g/L to about 12.0 g/L, about 12.0 g/L to about 13.3 g/L of resin, about 12.0 g/L to about 13.0 g/L, or about 13.0 g/L to about 13.3 g/L). After loading, the column can be washed with 3 CV of equilibration buffer. Following the wash phase, the column can be eluted using a linear gradient of decreasing pH and ionic strength. The gradient can be from 150 mM sodium acetate, pH 4.8-5.2 to 100 mM acetic acid over 20 CV. During elution, the column can be operated at a residence time of 10 minutes/CV.

In-process pool collection is achieved by fractionation of the product peak. All fractions are collected in sterile single use bags. The start and end of pooling is based on absorbance at 280 nm. When the UV₂₈₀ peak reaches ≥0.2 AU/cm, two 10-L fractions of eluate are collected prior to the collection of the main product fraction. The main product eluate pool collection stops when the UV₂₈₀ value reaches 55-60% of the peak maximum UV₂₈₀ value. Once the peak maximum is known, the contents of 10-L fractions with initial (at the start of fraction collection) UV₂₈₀ values above 15% of peak maximum are combined with the main product fraction after the elution is complete.

After elution, the column can be flushed with 3 CV of 100 mM acetic acid. After each use, the column can be cleaned with 3 CV of denaturing alkaline solution, 1.0 M NaOH, and held for a minimum sanitization duration of 30 minutes at 20° C. It can then be flushed with 3 CV of 0.1 M NaOH and stored until the next use. Exemplary process parameters for the Mixed Mode Chromatography step are provided in Table 11 below.

TABLE 11

| | Unit of | Normal Operating |
|---|---|---|
| Process Parameter | Measure | Range |
| Load factor | g/L resin | 5.1-13.3 |
| Process pH (wash/elution) | pH | 4.8-5.2 |
| Bed height | cm | 13-17 |
| Residence time (load/wash) | min/CV | 6.6-7.4 |
| Residence time (elution) | min/CV | 9.5-10.5 |
| Start collect | UV₂₈₀% peak | ≥15 |
| | maximum | |
| Stop collect | UV₂₈₀% peak | 55-60 |
| | maximum | |
| Temperature | ° C. | 17-23 |
| Gradient length | CV | 19.0-21.0 |
| Pool hold temperature | ° C. | 17-23 |
| Pool hold duration | hh:mm | ≤06:37 |

### Example 12: Ultrafiltration/Diafiltration 1 (UF/DF 1)

The UF/DF 1 step can be performed at 20° C. The Mixed Mode Chromatography pool is processed across a regenerated cellulose membrane (e.g. EMD Millipore Pellicon 2). Prior to use, the storage solution can be flushed out of the UF/DF 1 system with WFI until a conductivity of ≤10 μS/cm is reached. The membranes are then sanitized by circulating 0.1 N NaOH through the system for a minimum of 60 minutes. The sodium hydroxide is removed with a WFI flush until a conductivity of ≤2.1 μS/cm is reached, and equilibrated with diafiltration buffer (100 mM sodium acetate, pH 5.0).

The Mixed Mode Chromatography pool can be 0.2-μm filtered in-line and loaded on to the membrane at 42 g/m² and concentrated to 5.0 g/L at a transmembrane pressure (TMP) of 15 psig and a feed flow rate in the range of 27.7-33.9 L/min. The retentate can then be buffer exchanged with greater than 4.5 diavolumes of diafiltration buffer (100 mM sodium acetate, pH 5.0) and a permeate pH of 5.0, using the same parameters for TMP and feed flow rate as in the concentration phase. After arriving at a final retentate concentration, the UF/DF 1 pool can then be filtered through a 0.2-μm filter into a sterile single use bag.

Following usage, the membranes are flushed with WFI until a conductivity of ≤10 μ/cm is reached, and then cleaned and sanitized by circulating alkaline solution (0.1 N NaOH) though the UF/DF 1 system for a minimum of 60 minutes. The sanitization solution can be removed with a WFI flush until a conductivity of ≤2.1 μS/cm is reached, and the system can be stored in 0.1 N NaOH until the next use. Exemplary UF/DF 1 CPPs are provided in Table 12 below.

TABLE 12

| | Unit of | Normal Operating |
|---|---|---|
| Parameter | Measure | Range |
| Feed flow rate (concentration/ | L/min | 27.7-33.9 |
| diafiltration) | | |
| Membrane area | m² | N/A |
| Transmembrane Pressure | psi | 12-18 |
| (concentration/diafiltration) | | |
| Temperature | ° C. | 17-23 |
| Target product concentration | g/L | 4.5-5.5 |
| Diavolumes | N/A | ≥4.5 |
| Permeate pH | pH | 4.6-5.4 |
| Pool hold temperature | ° C. | 17-23 |
| Pool hold duration | hh:mm | ≤46:25 |

### Example 13: PEGylation

During the PEGylation step, an aldehyde modified PEG molecule (mPEG-aldehyde) is covalently attached to the N-terminus of the r-met-Hu-G-CSF protein to form PEGylated r-met-Hu-G-CSF. The protein is coupled with PEG, followed by reduction with sodium cyanoborohydride at a concentration of 20 mM to form a stable covalent bond between the PEG and protein.

The process step begins by preparing the PEGylation stock solution in a sterile single-use bag that is used as the reaction vessel. The PEGylation stock solution is a mixture of 50 g/L mPEG-aldehyde in UF/DF 1 diafiltration buffer (100 mM sodium acetate, pH 5.0). The UF/DF 1 product pool is then added to the vessel and the protein-PEG-aldehyde solution is allowed to mix for at least 15 minutes. Then, a 5 M sodium cyanoborohydride stock solution is added to achieve a final cyanoborohydride concentration of 20 mM, and the combined solution is mixed in the range of 17−23° C. and within a pH 4.7-5.3 for 3.75-4.25 hours.

After the specified reaction time, the reaction mixture can be added to WFI to dilute the mixture and slow the reaction. 1 L of reaction mixture can be added for every 3 L of WFI. The dilution can be performed using a sterile single-use bag as the holding vessel, and liquid transfer can be performed using a peristaltic pump. In some embodiments, the diluted pool can be immediately forward processed to the Cation Exchange Chromatography 2 step. Exemplary process parameters for the PEGylation are provided in Table 13 below.

TABLE 13

| | Unit of | Normal Operating |
|---|---|---|
| Process Parameter | Measure | Range |
| Reaction pH | pH | 4.8-5.2 |
| PEG:protein ratio | g/g | 4.7-5.3 |
| Reaction time (from sodium | hr | 3.75-4.25 |
| cyanoborohydride stock | | |
| solution addition to | | |
| addition of WFI) | | |
| Reaction temperature | ° C. | 17-23 |
| Reaction agitation speed | rpm | 240-260 |

### Example 14: Cation Exchange Chromatography 2

The Cation Exchange Chromatography 2 step purifies the PEGylated protein by removing PEGylation variants present in the PEGylation pool. The cation exchange resin in the chromatography system is packed in a 60-cm diameter column with a bed height of 25 cm. The temperature throughout this process step can be 20° C. Prior to each use, the column can be flushed with 2 CV of 0.1 N NaOH and sanitized with 3 CV of 1.0 N NaOH with a minimum sanitization time of 30 minutes. It can then be flushed with 2 CV of 0.1 N NaOH. The pre-use sanitization phases can be run at a residence time of 7.8 min/CV.

To facilitate equilibration, the sanitized column can be pre-equilibrated with 1.5 CV of 1 M sodium acetate, 1 M NaCl, pH 5.2-5.6 buffer. The equilibration phase can include a 3 CV flush of the column with 20 mM sodium acetate, pH 5.2-5.6. Both pre-equilibration and equilibration can be run at a flow rate equivalent of a 7.8 min/CV residence time. After equilibration, the diluted PEGylation pool can be 0.2-μm filtered in-line prior to loading. The diluted pool can be loaded onto the column at a load factor in the range of about 1.6 g/L to about 4.4 g/L (e.g., about 1.6 g/L to about 4.0 g/L, about 1.6 g/L to about 3.5 g/L, about 1.6 g/L to about 3.0 g/L, about 1.6 g/L to about 2.5 g/L, about 1.6 g/L to about 2.0 g/L, about 2.0 g/L to about 4.4 g/L, about 2.0 g/L to about 4.0 g/L, about 2.0 g/L to about 3.5 g/L, about 2.0 g/L to about 3.0 g/L, about 2.0 g/L to about 2.5 g/L, about 2.5 g/L to about 4.4 g/L, about 2.5 g/L to about 4.0 g/L, about 2.5 g/L to about 3.5 g/L, about 2.5 g/L to about 3.0 g/L, about 3.0 g/L to about 4.4 g/L, about 3.0 g/L to about 4.0 g/L, about 3.0 g/L to about 3.5 g/L, about 3.5 g/L to about 4.4 g/L, about 3.5 g/L to about 4.0 g/L, or about 4.0 g/L to about 4.4 g/L) of resin. The residence time during the load, wash, and elution phases can be 16.0 min/CV.

After loading, the column is washed with 3 column volumes of the 20 mM sodium acetate, pH 5.2-5.6 buffer. The column is then eluted using a linear gradient of increasing ionic strength. The gradient can be from 20 mM sodium acetate, pH 5.4 to 20 mM sodium acetate, 167 mM NaCl, pH 5.2-5.6, over 8.3-8.5 column volumes. In-process pool collection is achieved by fractionation of the product peak. All fractions can be collected in sterile single-use bags. The start and end of pooling is based on absorbance at 280 nm. When the UV₂₈₀ peak reaches ≥0.2 AU/cm, two 6-L fractions of eluate are collected, prior to the collection of the main product fraction. The main product eluate pool collection stops when the UV₂₈₀ value reaches 30-40% of the peak maximum UV₂₈₀ value. Once the peak maximum is known, the contents of 6-L fractions with initial (at the start of fraction collection) UV₂₈₀ values above 75% of peak maximum are combined with the main product fraction after the elution is complete.

The in-process pool is then conditioned with 0.1M HCl to a ratio of 0.135 kg HCl/kg pool. The column can be cleaned with 2 CV of a non-denaturing solution, 1.0 M NaCl and followed by 3 CV a denaturing solution, 1.0 N NaOH, with a minimum sanitization duration of 30 minutes at 20° C., before it is flushed with 3 CV of storage solution (0.1 N NaOH) and stored until the next use. Exemplary process parameters for the Cation Exchange Chromatography 2 step are provided in Table 14 below.

TABLE 14

| | Unit of | Normal Operating |
|---|---|---|
| Process Parameter | Measure | Range |
| Load factor | g/L resin | 1.6-4.4 |
| Process pH (wash/elution) | pH | 5.2-5.6 |
| Bed height | cm | 23-27 |
| Residence Time (load/ | min/CV | 14.4-17.6 |
| wash/elution) | | |
| Temperature | ° C. | 17-23 |
| Gradient length | CV | 8.3-8.5 |
| Start collect | UV₂₈₀% peak | ≥75 |
| | maximum | |
| Stop collect | UV₂₈₀% peak | 30-40 |
| | maximum | |
| Pool hold temperature | ° C. | 17-23 |
| Pool hold duration | hh:mm | ≤8:00 |

### Example 15: Ultrafiltration and Diafiltration 2 (UF/DF 2)

This process step can be performed at 20° C. The Cation Exchange Chromatography 2 pool can be processed across a regenerated cellulose membrane (e.g. EMD Millipore Pellicon 2). Prior to use, the storage solution can be flushed out of the UF/DF 2 system with WFI until a conductivity of ≤2.1 μS/cm is reached. The membranes can then be sanitized by circulating 0.1 N NaOH through the system for a minimum of 60 minutes. The sodium hydroxide can be removed with a WFI flush until a conductivity of ≤2.1 μS/cm is reached, and equilibrated with diafiltration buffer (10 mM acetate, 5% sorbitol, pH 4.0).

The Cation Exchange Chromatography pool can be 0.2-μm filtered in-line into the retentate circulation container. It can be loaded on to the membrane at 29 g/m² and concentrated to 8 g/L at 15 psig transmembrane pressure and a feed flow rate of 19.0 L/min. The retentate can then be buffer exchanged with 8.0 diavolumes of diafiltration buffer (10 mM acetate, 5% sorbitol, pH 4.0) using the same values for TMP and feed flow rate. The permeate pH at the end of diafiltration can be pH 4.0. The diafiltered pool can be further concentrated to 12.0 g/L in preparation for formulation. The final retentate can be transferred from the retentate vessel to a single-use bag using a peristaltic pump. The UF/DF 2 pool may be held at 20° C. for up to 8 hours prior to filling. Following usage, the membranes can be flushed with WFI until a conductivity of ≤10 μS/cm is reached, and then cleaned and sanitized by circulating alkaline solution (0.1 N NaOH) though the UF/DF 2 system for a minimum of 60 minutes. The sanitization solution can be removed with a WFI flush until a conductivity of ≤2.1 μS/cm is reached, and the system can be stored in 0.1 N NaOH until the next use. Exemplar Process Parameters for the UF/DF 2 step are provided in Table 15 below.

TABLE 15

| | Unit of | Normal Operating |
|---|---|---|
| Parameter | Measure | Range |
| Feed flow rate (concentration/ | L/min | 17.1-20.9 |
| diafiltration) | | |
| Membrane area | m2 | N/A |
| Transmembrane Pressure | psi | 13-17 |
| (concentration/diafiltration) | | |
| Temperature | ° C. | 17-23 |
| Target product concentration | g/L | 11.7-12.3 |
| Diavolumes | N/A | ≥7.5 |
| Permeate pH | pH | 3.6-4.4 |
| Pool hold temperature | ° C. | 17-23 |
| Pool hold duration | hh:mm | ≤08:00 |

### Example 16: Formulation and Fill

The UF/DF 2 pool is conditioned to achieve the PEGylated r-met-Hu-G-CSF formulation. The UF/DF 2 pool remains in a single-use bag and is supplemented with a calculated amount of solution of 10% polysorbate-20, 10 mM sodium acetate, and 5% sorbitol, pH 4.0 to achieve a final polysorbate-20 concentration of 0.004% (w/v). The supplemented pool undergoes pH adjustment using a calculated amount of titrant. The titrant is prepared from HCl that is diluted to a concentration of 0.1 M with 10 mM sodium acetate, 5% sorbitol, pH 4.0 (diafiltration buffer). The UF/DF 2 pool can then be further diluted with additional diafiltration buffer to achieve a protein concentration of 10.0 mg/mL.

The DS fill can be performed in an ISO 5 laminar flow hood. The formulated PEGylated r-met-Hu-G-CSF can be 0.22-μm filtered into clean certified, sterile polyethylene terephthalate glycol-modified (PETG) bottles for storage. 250 mL of formulated product can be flushed to waste, and then 1,000-mL and 250-mL bottles are filled to within the cylindrical section of the bottleneck to minimize headspace. The caps of each bottle are torqued to 30±3 in·lbs. The filter is integrity tested post-use. Exemplary process parameters for the Formulation and Fill step are provided in Table 16 below.

TABLE 16

| | Unit of | Normal Operating |
|---|---|---|
| Process Parameter | Measure | Range |
| 10% polysorbate-20 addition | mL | ±10% of |
| quantity | | calculated target |
| Titrant addition quantity | kg | ±5% of |
| | | calculated target |
| Buffer addition quantity | kg | ±3% of |
| | | calculated target |
| Formulated UF/DF 2 pool | min | 5-15 |
| mix time | | |
| Formulated UF/DF 2 pool | N/A a | N/A a |
| mix speed | | |
| Volume of buffer consumed | L | ≥2 |
| for pre-use filter wetting | | |
| Waste flush volume | mL | 240-260 |
| Filter loading | g/cm² | ≤0.19 |

### Other Embodiments

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for producing recombinant methionyl human granulocyte colony-stimulating factor (r-met-Hu-G-CSF) comprising:
(a) contacting cells comprising a nucleic acid encoding r-met-Hu-G-CSF with a culture medium to create a fermentation medium;
(b) fermenting the cells under fed-batch conditions causing the cells to produce r-met-Hu-G-CSF;
(c) harvesting the cells from the fermentation medium by centrifugation;
(d) lysing the cells harvested from the fermentation medium to release inclusion bodies comprising r-met-Hu-G-CSF; and
(e) storing the inclusion bodies.

2. A method for purifying r-met-Hu-G-CSF from inclusion bodies comprising:
(a) suspending inclusion bodies comprising r-met-Hu-G-CSF in a solubilization buffer;
(b) oxidizing solubilized r-met-Hu-G-CSF to permit the r-met-Hu-G-CSF to fold and form disulfide bonds;
(c) subjecting a product of step (b) to Dowex flow-through chromatography;
(d) subjecting a product of step (c) to acid precipitation;
(e) subjecting a product of step (d) to anion exchange chromatography;
(f) subjecting a product of step (e) to cation exchange chromatography;
(g) subjecting a product of step (f) to mixed mode chromatography;
(h) concentrating a product of step (g); and
(i) exchanging r-met-Hu-G-CSF in the product of step (h) into a buffer by ultrafiltration and diafiltration.

3. A method of producing a PEGylated and purified r-met-Hu-G-CSF comprising:
(a) contacting a r-met-Hu-G-CSF with a PEGylation reagent under suitable reaction conditions to PEGylate the r-met-Hu-G-CSF;
(b) subjecting a product of step (a) to cation exchange chromatography to remove the reaction by-products from PEGylated r-met-Hu-G-CSF;
(c) concentrating a product of step (b);
(d) exchanging the PEGylated r-met-Hu-G-CSF in a product of step (c) into a buffer by ultrafiltration and diafiltration;
(e) adding a surfactant to a product of step (d);
(f) adjusting the pH of a product of step (e) to a target value by adding HCl or NaOH;
(g) diluting a product of step (f) with additional diafiltration buffer to achieve a target PEGylated r-met-Hu-G-CSF concentration of 10.0 mg/mL; and
(h) subjecting the PEGylated r-met-Hu-G-CSF product of step (g) to 0.2-μm filtration.

4. The method of claim 3, further comprising, after step (h), storing the PEGylated r-met-Hu-G-CSF product at 5±3° C.

5. The method of claim 1, further comprising after step (e):
(f) suspending inclusion bodies comprising r-met-Hu-G-CSF in a solubilization buffer;
(g) oxidizing solubilized r-met-Hu-G-CSF to permit the r-met-Hu-G-CSF to fold and form disulfide bonds;
(h) subjecting a product of step (g) to Dowex flow-through chromatography;
(i) subjecting a product of step (h) to acid precipitation;
(j) subjecting a product of step (i) to anion exchange chromatography;
(k) subjecting a product of step (j) to cation exchange chromatography;
(l) subjecting a product of step (k) to mixed mode chromatography;
(m) concentrating a product of step (l); and
(n) exchanging r-met-Hu-G-CSF in the product of step (m) into a buffer by ultrafiltration and diafiltration.

6. The method of 2, further comprising after step (i):
(j) contacting a r-met-Hu-G-CSF with a PEGylation reagent under suitable reaction conditions to PEGylate the r-met-Hu-G-CSF;
(k) subjecting a product of step (j) to cation exchange chromatography to remove the reaction by-products from PEGylated r-met-Hu-G-CSF;
(l) concentrating a product of step (k);
(m) exchanging the PEGylated r-met-Hu-G-CSF in a product of step (l) into a buffer by ultrafiltration and diafiltration;
(n) adding a surfactant to a product of step (m);
(o) adjusting the pH of a product of step (n) to a target value by adding HCl or NaOH;
(p) diluting a product of step (o) with additional diafiltration buffer to achieve a target PEGylated r-met-Hu-G-CSF concentration of 10.0 mg/mL; and
(q) subjecting the PEGylated r-met-Hu-G-CSF product of step (p) to 0.2-μm filtration.

7. The method of claim 5, further comprising after step (n):
(o) contacting a r-met-Hu-G-CSF with a PEGylation reagent under suitable reaction conditions to PEGylate the r-met-Hu-G-CSF;
(p) subjecting a product of step (o) to cation exchange chromatography to remove the reaction by-products from PEGylated r-met-Hu-G-CSF;
(q) concentrating a product of step (p);
(r) exchanging the PEGylated r-met-Hu-G-CSF in a product of step (q) into a buffer by ultrafiltration and diafiltration;
(s) adding a surfactant to a product of step (r);
(t) adjusting the pH of a product of step (s) to a target value by adding HCl or NaOH;
(u) diluting a product of step (t) with additional diafiltration buffer to achieve a target PEGylated r-met-Hu-G-CSF concentration of 10.0 mg/mL; and
(v) subjecting the PEGylated r-met-Hu-G-CSF product of step (u) to 0.2-μm filtration.

8. The method of claim 7, further comprising storing the PEGylated r-met-Hu-G-CSF product at 5±3° C.
